(19) United States
(12) Patent Application Publication
(10) Pub. No.: US 2005/0104042 A1
(43) Pub. Date: May 19, 2005

(54) Process for the synthesis of amine ethers from secondary amino oxides

(76) Inventors: Markus Frey, Rheinfelden (CH); Valerie Rast, Basel (CH)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 10/496,773

(22) PCT Filed: Nov. 19, 2002
(86) PCT No.: PCT/EP02/12957

(30) Foreign Application Priority Data
Nov. 26, 2001 (EP) 01811143.5

(51) Int. Cl. C09K 15/04; C07C 215/00

(52) U.S. Cl. 252/399; 564/503

(57) ABSTRACT

Amine ethers of sterically hindered amines are obtained in good yield from the corresponding N-oxyl hindered amine precursor by reaction with a hydrocarbon in the presence of an organic hydroperoxide and an iodide. The products of present process find utility as polymerization regulators and/or light stabilizers for organic material.

The instant invention pertains to a process for preparing amine ethers, e.g. N-hydrocarbyloxy substituted hindered amine compounds, by the reaction of the corresponding N-oxyl intermediate with a hydrocarbon in presence of an organic hydroperoxide and an iodide catalyst.

4-Hydroxy-1-oxyl-2,2,6,6-tetramethylpiperidine and 4-oxo-1-oxyl-2,2,6,6-tetramethylpiperidine are described as scavengers for some carbon centered radicals (S. Nigam et al., J. Chem. Soc., Trans. Faraday Soc., 1976, (72), 2324 and by K.-D. Asmus et al., Int. J. Radiat. Biol., 1976, (29), 211).

D. H. R. Barton et al., Tetrahedron, 1996, (52), 10301 describe the formation of some N-alkoxy-2,2,6,6-tetramethylpiperidine derivatives in the reaction of hydrocarbons with iron(II) and iron(III) species, hydrogen peroxide and various coadditives in the presence of N-oxyl-2,2,6,6-tetramethylpiperidine (TEMPO).

U.S. Pat. No. 5,374,729 describes a process for the preparation of N-methoxy derivatives of hindered amines from the reaction of the corresponding N-oxyl compound with methyl radicals produced from dimethyl sulfoxide by decomposing aqueous hydrogen peroxide in presence of a metal salt or by thermal decomposition of di-tert.butyl peroxide.

U.S. Pat. No. 4,921,962 describes a process for the formation of N-hydrocarbyloxy derivatives of sterically hindered amines in which a hindered amine or N-oxyl substituted hindered amine is reacted with a hydrocarbon solvent in the presence of a hydroperoxide and a molybdenum catalyst.

It has now been found that N-hydrocarbyloxy substituted sterically hindered amines can most suitably be prepared from the N-oxyl intermediate and a hydrocarbon in presence of an organic hydroperoxide and an iodide catalyst. The process of the invention uses only catalytic quantities of iodide and does not require high temperatures.

Thus, present invention pertains to a process for the preparation of an amine ether of a sterically hindered amine by reacting a corresponding sterically hindered aminoxide with an aliphatic hydrocarbon compound, characterized in that the reaction is carried out in the presence of an organic hydroperoxide and an iodide, which is preferably used in a catalytic amount.

The aliphatic hydrocarbon compound may be any compound selected from alkane, alkene, alkyne, or cyclic or polycyclic analogues thereof, and optionally may be substituted, e.g. by aryl, halogen, alkoxy etc., provided that an aliphatic CH (or CH₂, CH₃) moiety is contained.

Advantageously, the process of the invention is carried out in the absence of a copper or a copper compound, preferably in the absence of any heavy metal or heavy metal compound. Heavy metal is to be understood as transition metal or any metal of higher molecular weight than calcium. Metal compounds, the presence of which is advantageously to be avoided in the present process, include any form like salts, complexes, solutions and dispersions thereof. The amounts of these compounds to be tolerated within the process of the invention are preferably well below the catalytic level, e.g. below 0.0001 molar equivalent per mole of nitroxyl moiety, more preferably within or below the ppm-level (up to 1000 parts by weight of heavy metal per 1 million parts by weight of total reaction mixture).

Preferred is a process for the preparation of an amine ether of the formula A 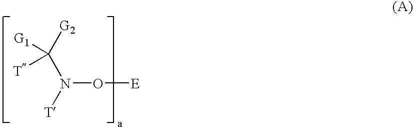
wherein
a is 1 or 2;
when a is 1, E is E′
when a is 2, E is L;

E′ is C₁-C₃₆ alkyl; C₃-C₁₈ alkenyl; C₂-C₁₈ alkinyl; C₅-C₁₈ cycloalkyl; C₅-C₁₈ cycloalkenyl; a radical of a saturated or unsaturated aliphatic bicyclic or tricyclic hydrocarbon of 7 to 12 carbon atoms; C₂-C₇alkyl or C₃-C₇alkenyl substituted by halogen, C₁-C₈alkoxy or phenoxy; C₄-C₁₂heterocycloalkyl; C₄-C₁₂heterocycloalkenyl; C₇-C₁₅ aralkyl or C₄-C₁₂heteroaralkyl, each of which is unsubstituted or substituted by C₁-C₄ alkyl or phenyl; or E′ is a radical of formula (VII) or (VIII) 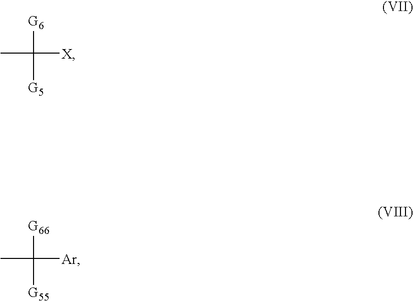
Ar is C₆-C₁₀aryl or C₅-C₉heteroaryl;
X is phenyl, naphthyl or biphenyl, which are substituted by 1, 2, 3 or 4 D and optionally further substituted by NO₂, halogen, amino, hydroxy, cyano, carboxy, C₁-C₄alkoxy, C₁-C₄alkylthio, C₁-C₄alkylamino or di(C₁-C₄alkyl)amino;
D is a group 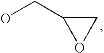
   a group C(O)-G₁₃ or a group C(O)-G₉-C(O)-G₁₃;
G₁ and G₂, independently of each other, are hydrogen, halogen, NO₂, cyano, —CONR₅R₆, —(R₉)COOR₄, —C(O)—R₇, —OR₈, —SR₈, —NHR₈, —N(R₁₈)₂, carbamoyl, di(C₁-C₁₈alkyl)carbamoyl, —C(═NR₅)(NHR₆), C₁-C₁₈alkyl; C₃-C₁₈alkenyl; C₃-C₁₈alkinyl, C₇-C₉phenylalkyl, C₃-C₁₂cycloalkyl or C₂-C₁₂heterocycloalkyl; C₁-C₁₈alkyl or C₃-C₁₈alkenyl or C₃-C₁₈alkinyl or C₇-C₉phenylalkyl, C₃-C₁₂cycloalkyl or C₂-C₁₂heterocycloalkyl substituted by OH, halogen, NO₂, amino, cyano, carboxy, COOR₂₁, C(O)—R₂₂, C₁-C₄alkoxy, C₁-C₄alkylthio, C₁-C₄alkylamino or di(C₁-C₄alkyl)amino or a group —O—C(O)—R₇; C₂-C₁₈alkyl which is interrupted by at least one O atom and/or NR₅ group; or are C₆-C₁₀aryl; or phenyl or naphthyl which are substituted by C₁-C₄alkyl, C₁-C₄alkoxy, C₁-C₄alkylthio, halogen, cyano, hydroxy, carboxy, COOR₂₁, C(O)—R₂₂, C₁-C₄alkylamino or di(C₁-C₄alkyl)amino; or G₁ and G₂ together with the linking carbon atom form a C₃-C₁₂cycloalkyl radical;
G₅ and G₆ are independently of each other H or CH₃;
G₉ is C₁-C₁₂alkylene or a direct bond;
G₁₃ is C₁-C₁₈alkyl;
G₁₄ is C₁-C₁₈alkyl, C₅-C₁₂cycloalkyl, an acyl radical of an aliphatic or unsaturated aliphatic carboxylic or carbamic acid containing 2 to 18 carbon atoms, an acyl radical of a cycloaliphatic carboxylic or carbamic acid containing 7 to 12 carbon atoms, or acyl radical of an aromatic acid containing 7 to 15 carbon atoms;
G₅₅ is H, CH₃ or phenyl;
G₆₆ is —CN or a group of the formula —COOR₄ or —CONR₅R₆ or —CH₂—O-G₁₄;
L is alkylene of 1 to 18 carbon atoms, cycloalkylene of 5 to 8 carbon atoms, cycloalkenylene of 5 to 8 carbon atoms, alkenylene of 3 to 18 carbon atoms, alkylene of 1 to 12 carbon atoms substituted by phenyl or by phenyl substituted by alkyl of 1 to 4 carbon atoms; or is alkylene of 4 to 18 carbon atoms interrupted by COO and/or phenylene;
T′ is tertiary C₄-C₁₈alkyl or phenyl, each of which are unsubstituted or substituted by halogen, OH, COOR₂₁ or C(O)—R₂₂; or T′ is C₅-C₁₂cycloalkyl; C₅-C₁₂cycloalkyl which is interrupted by at least one O or —NR₁₈—; a polycyclic alkyl radical having 7-18 carbon atoms, or the same radical which is interrupted by at least one O or —NR₁₈—; or T′ is —C(G₁)(G₂)-T″; or C₁-C₁₈alkyl or C₅-C₁₂cycloalkyl substituted by 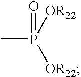
T″ is hydrogen, halogen, NO₂, cyano, or is a monovalent organic radical comprising 1-50 carbon atoms;
or T″ and T′ together form a divalent organic linking group completing, together with the hindered amine nitrogen atom and the quaternary carbon atom substituted by G₁ and G₂, an optionally substituted five- or six-membered ring structure;
   and
R₄ is hydrogen, C₁-C₁₈alkyl, phenyl, an alkali metal cation or a tetraalkylammonium cation;
R₅ and R₆ are hydrogen, C₁-C₁₈alkyl, C₂-C₁₈alkyl which is substituted by hydroxy or, taken together, form a C₂-C₁₂alkylene bridge or a C₂-C₁₂-alkylene bridge interrupted by O or/and NR₁₈;
R₇ is hydrogen, C₁-C₁₈alkyl or C₆-C₁₀aryl;
R₈ is hydrogen, C₁-C₁₈alkyl or C₂-C₁₈hydroxyalkyl;
R₉ is C₁-C₁₂alkylene or a direct bond;
R₁₈ is C₁-C₁₈alkyl or phenyl, which are unsubstituted or substituted by halogen, OH, COOR₂₁ or C(O)—R₂₂;
R₂₁ is hydrogen, a alkali metal atom or C₁-C₁₈alkyl; and
R₂₂ is C₁-C₁₈alkyl;
   which process comprises

reacting a N-oxyl amine of formula B 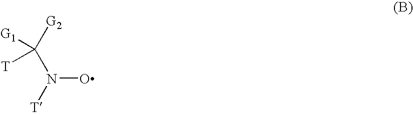
with a compound of formula IV or V
E′-H (IV)
H-L-H (V)
in the presence of an organic hydroperoxide and a catalytic amount of an iodide.

More specifically, present invention pertains to a process for the preparation of an amine ether of the formula A 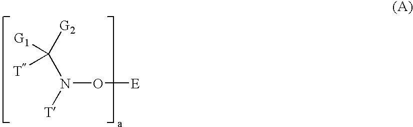
wherein
a is 1 or 2;
when a is 1, E is E′
when a is 2, E is L;
E′ is C₁-C₃₆ alkyl; C₃-C₁₈ alkenyl; C₂-C₁₈ alkinyl; C₅-C₁₈ cycloalkyl; C₅-C₁₈ cycloalkenyl; a radical of a saturated or unsaturated aliphatic bicyclic or tricyclic hydrocarbon of 7 to 12 carbon atoms; C₂-C₇alkyl or C₃-C₇alkenyl substituted by halogen; C₇-C₁₅ aralkyl or C₇-C₁₅ aralkyl substituted by C₁-C₄ alkyl or phenyl; or E′ is a radical of formula (VII) 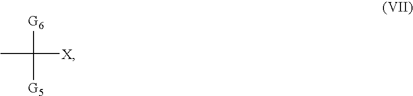
   wherein
X is phenyl, naphthyl or biphenyl, which are substituted by 1, 2, 3 or 4 D and optionally further substituted by NO₂, halogen, amino, hydroxy, cyano, carboxy, C₁-C₄alkoxy, C₁-C₄alkylthio, C₁-C₄alkylamino or di(C₁-C₄alkyl)amino;
D is a group 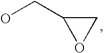
   a group C(O)-G₁₃ or a group C(O)-G₉-C(O)-G₁₃;
G₁ and G₂, independently of each other, are hydrogen, halogen, NO₂, cyano, —CONR₅R₆, —(R₉)COOR₄, —C(O)—R₇, —OR₈, —SR₈, —NHR₈, —N(R₁₈)₂, carbamoyl, di(C₁-C₁₈alkyl)carbamoyl, —C(═NR₅)(NHR₆), C₁-C₁₈alkyl; C₃-C₁₈alkenyl; C₃-C₁₈alkinyl, C₇-C₉phenylalkyl, C₃-C₁₂cycloalkyl or C₂-C₁₂heterocycloalkyl; C₁-C₁₈alkyl or C₃-C₁₈alkenyl or C₃-C₁₈alkinyl or C₇C₉phenylalkyl, C₃-C₁₂cycloalkyl or C₂-C₁₂heterocycloalkyl substituted by OH, halogen, NO₂, amino, cyano, carboxy, COOR₂₁, C(O)—R₂₂, C₁-C₄alkoxy, C₁-C₄alkylthio, C₁-C₄alkylamino or di(C₁-C₄alkyl)amino or a group —O—C(O)—R₇; C₂-C₁₈alkyl which is interrupted by at least one O atom and/or NR₅ group; or are C₆-C₁₀aryl; or phenyl or naphthyl which are substituted by C₁-C₄alkyl, C₁-C₄alkoxy, C₁-C₄alkylthio, halogen, cyano, hydroxy, carboxy, COOR₂₁, C(O)—R₂₂, C₁-C₄alkylamino or di(C₁-C₄alkyl)amino; or G₁ and G₂ together with the linking carbon atom form a C₃-C₁₂cycloalkyl radical;
G₅ and G₆ are independently of each other H or CH₃;
G₉ is C₁-C₁₂alkylene or a direct bond;
G₁₃ is C₁-C₁₈alkyl;
L is alkylene of 1 to 18 carbon atoms, cycloalkylene of 5 to 8 carbon atoms, cycloalkenylene of 5 to 8 carbon atoms, alkenylene of 3 to 18 carbon atoms, alkylene of 1 to 12 carbon atoms substituted by phenyl or by phenyl substituted by alkyl of 1 to 4 carbon atoms;
T′ is tertiary C₄-C₁₈alkyl or phenyl, each of which are unsubstituted or substituted by halogen, OH, COOR₂₁ or C(O)—R₂₂; or T′ is C₅-C₁₂cycloalkyl; C₅-C₁₂cycloalkyl which is interrupted by at least one O or —NR₁₈—; a polycyclic alkyl radical having 7-18 carbon atoms, or the same radical which is interrupted by at least one O or —NR₁₈—; or T′ is —C(G₁)(G₂)-T″; or C₁-C₁₈alkyl or C₅-C₁₂cycloalkyl substituted by 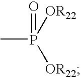
T′ is hydrogen, halogen, NO₂, cyano, or is a monovalent organic radical comprising 1-50 carbon atoms;
or T″ and T′ together form a divalent organic linking group completing, together with the hindered amine nitrogen atom and the quaternary carbon atom substituted by G₁ and G₂, an optionally substituted five- or six-membered ring structure;
   and
R₄ is hydrogen, C₁-C₁₈alkyl, phenyl, an alkali metal cation or a tetraalkylammonium cation;
R₅ and R₆ are hydrogen, C₁-C₁₈alkyl, C₂-C₁₈alkyl which is substituted by hydroxy or, taken together, form a C₂-C₁₂alkylene bridge or a C₂-C₁₂-alkylene bridge interrupted by O or/and NR₁₈—;
R₇ is hydrogen, C₁-C₁₈alkyl or C₆-C₁₀aryl;
R₈ is hydrogen, C₁-C₁₈alkyl or C₂-C₁₈hydroxyalkyl;
R₉ is C₁-C₁₂alkylene or a direct bond;
R₁₈ is C₁-C₁₈alkyl or phenyl, which are unsubstituted or substituted by halogen, OH, COOR₂₁ or C(O)—R₂₂;
R₂₁ is hydrogen, a alkali metal atom or C₁-C₁₈alkyl; and
R₂₂ is C₁-C₁₈alkyl;
   which process comprises

reacting a N-oxyl amine of formula B 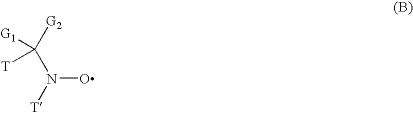
with a hydrocarbon of formula IV or V
E′-H (IV)
H-L-H (V)
in the presence of an organic hydroperoxide and a catalytic amount of an iodide.

In particular, present invention pertains to a process for the synthesis of a hindered amine of formula I or II 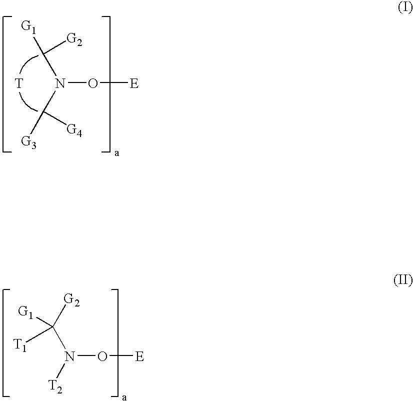
wherein
G₁, G₂, G₃ and G₄ independently of each other are C₁-C₁₈alkyl; C₃-C₁₈alkenyl; C₃-C₁₈alkinyl; C₁-C₁₈alkyl or C₃-C₁₈alkenyl or C₃-C₁₈alkinyl substituted by OH, halogen or a group —O—C(O)—R₅; C₂-C₁₈alkyl which is interrupted by at least one O atom and/or NR₅ group; or are C₃-C₁₂cycloalkyl; or C₆-C₁₀aryl; or G₁ and G₂ and/or G₃ and G₄ together with the linking carbon atom form a C₃-C₁₂cycloalkyl radical;
a is 1 or 2;
when a is 1, E is E′, wherein E′ is C₁-C₃₆ alkyl; C₂-C₁₈ alkenyl; C₂-C₁₈ alkinyl; C₅-C₁₈ cycloalkyl; C₅-C₁₈ cycloalkenyl; a radical of a saturated or unsaturated aliphatic bicyclic or tricyclic hydrocarbon of 7 to 12 carbon atoms; C₂-C₇alkyl or C₃-C₇alkenyl substituted by halogen; C₇-C₁₅ aralkyl or C₇-C₁₅ aralkyl substituted by C₁-C₄ alkyl or phenyl; or E′ is a radical of formula (VII) 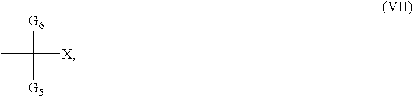
   wherein
X is phenyl, naphthyl or biphenyl, which are substituted by 1, 2, 3 or 4 D and optionally further substituted by NO₂, halogen, amino, hydroxy, cyano, carboxy, C₁-C₄alkoxy, C₁-C₄alkylthio, C₁-C₄alkylamino or di(C₁-C₄alkyl)amino;
D is a group 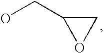
   a group C(O)-G₁₃ or a group C(O)-G₉-C(O)-G₁₃;
when a is 2, E is L;
G₅ and G₆ are independently of each other H or CH₃;
G₉ is C₁-C₁₂alkylene or a direct bond;
G₁₃ is C₁-C₁₈alkyl;
L is alkylene of 1 to 18 carbon atoms, cycloalkylene of 5 to 8 carbon atoms, cycloalkenylene of 5 to 8 carbon atoms, alkenylene of 3 to 18 carbon atoms, alkylene of 1 to 12 carbon atoms substituted by phenyl or by phenyl substituted by alkyl of 1 to 4 carbon atoms;
T is a divalent organic radical required to complete formula I to form, together with the hindered amine nitrogen atom and the two quaternary carbon atoms substituted by G₁ and G₂ or G₃ and G₄, a five- or six-membered ring structure;
T₁ is hydrogen, halogen, NO₂, cyano, —(R₉)COOR₄, —(R₉)C(O)—R₇, —OR₈, unsubstituted C₁-C₁₈alkyl, C₂-C₁₈alkenyl, C₂-C₁₈alkynyl, C₇C₉phenylalkyl, C₃-C₁₂cycloalkyl or C₂-C₁₂heterocycloalkyl; or T₁ is C₁-C₁₈alkyl, C₂-C₁₈alkenyl, C₂-C₁₈ alkynyl, C₇C₉phenylalkyl, C₃-C₁₂cycloalkyl or C₂-C₁₂heterocycloalkyl, which is substituted by NO₂, halogen, hydroxy, cyano, carboxy, C₁-C₆alkanoyl, C₁-C₁₂alkoxy; or phenyl, naphthyl, which are unsubstituted or substituted by C₁-C₄alkyl, C₁-C₄alkoxy, C₁-C₄alkylthio, halogen, cyano, hydroxy, carboxy; or T₁ is a residue —CH₂—O—R₁₀ or —CH₂—NR₁₈—R₁₀ or —C(═CH₂)—R₁₁ or —C(═O)—R₁₂;
T₂ is tertiary C₄-C₁₈alkyl or phenyl, which are unsubstituted or substituted by halogen, OH, COOR₂₁ or C(O)—R₂₂; or T₂ is C₅-C₁₂cycloalkyl; C₅-C₁₂cycloalkyl which is interrupted by at least one O; a polycyclic alkyl radical having 7-18 carbon atoms or the same radical which is interrupted by at least one O atom; or T₂ is —C(G₁)(G₂)-T₁; or 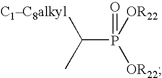
R₄ is hydrogen, C₁-C₁₈alkyl, phenyl, an alkali metal cation or a tetraalkylammonium cation;
R₅ is hydrogen, C₁-C₁₈alkyl or C₆-C₁₀aryl
R₇ is hydrogen, C₁-C₁₈alkyl or phenyl;
R₈ is hydrogen, C₁-C₁₈alkyl or C₂-C₁₈hydroxyalkyl;
R₉ is C₁-C₁₂alkylene or a direct bond;
R₁₀ is hydrogen, formyl, C₂-C₁₈alkylcarbonyl, benzoyl, C₁-C₁₈alkyl, C₅-C₁₂cycloalkyl, C₅-C₁₂cycloalkyl interrupted by O or NR₁₈, or is benzyl or phenyl which are unsubstituted or substituted by halogen, OH, COOR₂₁ or C(O)—R₂₂;
R₁₁ is OH, C₁-C₁₈alkoxy, benzyloxy, O—C(O)—(C₁-C₁)alkyl, N(R₁₈)₂, or a group C(O)R₂₅;
R₁₂ is OH, O(alkali-metal), C₁-C₁₈alkoxy, benzyloxy, N(R₁₈)₂;
R₁₈ is C₁-C₁₈alkyl or C₂-C₁₈hydroxyalkyl;
R₂₁ is hydrogen, a alkali metal atom or C₁-C₁₈alkyl; and
R₂₂ is C₁-C₁₈alkyl;
R₂₅ is OH, C₁-C₁₈alkoxy, benzyloxy, N(R₁₈)₂;
   which process comprises
   reacting a N-oxyl hindered amine of formula III or IIIa 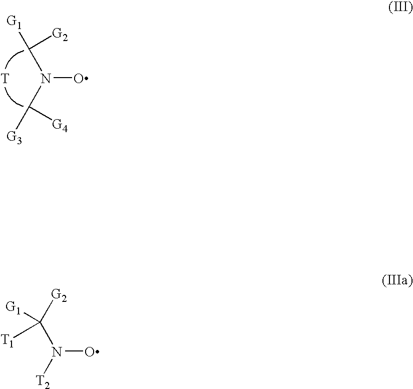
   with a hydrocarbon of formula IV or V
   E′-H (IV)
   H-L-H (V)
   in the presence of an organic hydroperoxide and a catalytic amount of an iodide.

In the context of the description of the present invention, the term alkyl comprises, for example, methyl, ethyl and the isomers of propyl, butyl, pentyl, hexyl, heptyl, octyl, nonyl, decyl, undecyl and dodecyl. Examples of aryl-substituted alkyl (aralkyl) are benzyl, α-methylbenzyl or cumyl. Examples of alkoxy are methoxy, ethoxy, propoxy, butoxy, octyloxy etc. Examples of alkenyl are vinyl and especially allyl. Examples of alkylene including alkylidene are ethylene, n-propylene or 1,2-propylene.

Some examples of cycloalkyl are cyclobutyl, cyclopentyl, cyclohexyl, methylcyclopentyl, dimethylcyclopentyl and methylcyclohexyl.

Examples of aryl are phenyl and naphthyl. Examples of substituted aryl are methyl-, dimethyl-, trimethyl-, methoxy- or phenyl-substituted phenyl.

Some examples of an aliphatic carboxylic acid are acetic, propionic, butyric, stearic acid. An example of a cycloaliphatic carboxylic acid is cyclohexanoic acid. An example of an aromatic carboxylic acid is benzoic acid. An example of a phosphorus-containing acid is methylphosphonic acid. An example of an aliphatic dicarboxylic acid is malonyl, maleoyl or succinyl, or sebacic acid. An example of a residue of an aromatic dicarboxylic acid is phthaloyl.

A group heterocycloalkyl or heterocycloalkenyl embraces one or two heteroatoms, and a group heteroaryl from one to four heteroatoms, the heteroatoms being preferably selected from the group consisting of nitrogen, sulfur and oxygen. Some examples of heterocycloalkyl are tetrahydrofuryl, pyrrolidinyl, piperazinyl and tetrahydrothienyl. Some examples of heteroaryl are furyl, thienyl, pyrrolyl, pyridyl and pyrimidinyl. C₂-C₁₂heterocycloalkyl is typically oxirane, 1,4-dioxane, tetrahydrofuran, γ-butyrolactone, ε-caprolactam, oxirane, aziridine, diaziridine, pyrrole, pyrrolidine, thiophen, furan, pyrazole, imidazole, oxazole, oxazolidine, thiazole, pyran, thiopyran, piperidine or morpholine.

An example of a monovalent silyl radical is trimethylsilyl.

Polycyclic alkyl radicals which may also be interrupted by at least one oxygen or nitrogen atom are for example adamantane, cubane, twistane, norbornane, bycyclo[2.2.2]octane bycyclo[3.2.1]octane, hexamethylentetramine (urotropine) or a group 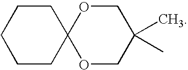
Acyl radicals of monocarboxylic acids are, within the definitions, a residue of the formula —CO—R″, wherein R″ may stand inter alia for an alkyl, alkenyl, cycloalkyl or aryl radical as defined. Preferred acyl radicals include acetyl, benzoyl, acryloyl, methacryloyl, propionyl, butyryl, valeroyl, hexanoyl, heptanoyl, octanoyl, nonanoyl, decanoyl, undecanoyl, dodecanoyl, pentadecanoyl, stearoyl. Polyacyl radicals of polyvalent acids are of the formula (—CO)ₙ—R″, wherein n is the valency, e.g. 2, 3, 4, 5 or 6. Some preferred examples for such residues are given elsewhere.

In preferred products of the instant process, E′ is selected from the group consisting of 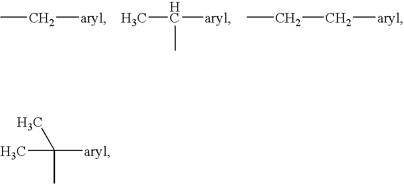
(C₅-C₆cycloalkyl)₂CCN, (C₁-C₁₂alkyl)₂CCN, —CH₂CH═CH₂, (C₁-C₁₂)alkyl-CR₃₀—C(O)—(C₁-C₁₂)alkyl, (C₁-C₁₂)alkyl-CR₃₀—C(O)—(C₆-C₁₀)aryl, (C₁-C₁₂)alkyl-CR₃₀—C(O)—(C₁-C₁₂)alkoxy, (C₁-C₁₂)alkyl-CR₃₀—C(O)-phenoxy, (C₁-C₁₂)alkyl-CR₃₀—C(O)—N-di(C₁-C₁₂)alkyl, (C₁-C₁₂)alkyl-CR₃₀—CO—NH(C₁-C₁₂)alkyl, (C₁-C₁₂)alkyl-CR₃₀—CO—NH₂, —CH₂CH═CH—CH₃, —CH₂—C(CH₃)═CH₂, —CH₂—CH═CH-phenyl, 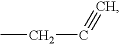
(C₁-C₁₂)alkyl-CR₃₀—CN, 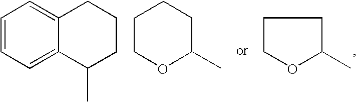
wherein
R₃₀ is hydrogen or C₁-C₁₂alkyl;
the aryl groups are phenyl or naphthyl, which are unsubstituted or substituted with C₁-C₁₂alkyl, halogen, C₁-C₁₂alkoxy, formyl, C₂-C₁₂alkylcarbonyl, glycidyloxy, OH, —COOH or —COOC₁-C₁₂alkyl. More preferably E′ is selected from the group consisting of —CH₂-phenyl, CH₃CH-phenyl, (CH₃)₂C-phenyl, (C₅-C₆cycloalkyl)₂CCN, (CH₃)₂CCN, —CH₂CH═CH₂, CH₃CH—CH═CH₂(C₁-C₈alkyl) CR₃₀—C(O)-phenyl, (C₁-C₈)alkyl-CR₃₀—C(O)—(C₁-C₈)alkoxy, (C₁-C₈)alkyl-CR₃₀—C(O)—(C₁-C₈)alkyl, (C₁-C₈)alkyl-CR₃₀—C(O)—N-di(C₁-C₈)alkyl, (C₁-C₈)alkyl-CR₃₀—C(O)—NH(C₁-C₈)alkyl, (C₁-C₈)alkyl-CR₃₀—C(O)—NH₂, (C₁-C₁₂)alkyl-CR₃₀—CN, wherein R₃₀ is hydrogen or (C₁-C₈)alkyl.

G₁ and G₂ and/or G₃ and G₄ forming, together with the linking carbon atom, a C₃-C₁₂cycloalkyl radical, preferably form a C₅-C₁₂cycloalkyl radical, especially cyclopentylene, cyclohexylene or cycloheptylene.

G₁, G₂, G₃ and G₄ independently are preferably alkyl of 1 to 4 carbon atoms, or the adjacent radicals G₁ and G₂ and/or G₃ and G₄ together are pentamethylene. More preferably, G₁, G₂, G₃ and G₄ independently are methyl or ethyl or propyl, especially methyl or ethyl. In the products most preferred, G₁ and G₃ are each methyl while G₂ and G₄ independently are methyl, ethyl or propyl.

T usually is an organic linking group containing 2-500 carbon atoms and forming, together with the carbon atoms it is directly connected to and the nitrogen atom, a substituted, 5-, 6 or 7-membered cyclic ring structure; T is preferably a C₂-C₅₀₀hydrocarbon optionally containing 1-200 hetero atoms selected from nitrogen, oxygen, phosphorus, sulfur, silicon and halogen, T therein can be part of a 6-membered cyclic ring structure. More preferably, T is an organic linking group of the formula 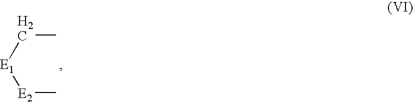
wherein
E₂ is —CO— or —(CH₂)_{b}—, while b is 0, 1 or 2;
E₁ is a carbon atom carrying the two residues R₂₄ and R₂₅, or is >N—R₂₅, or is oxygen, and R₂₄ and R₂₅ are hydrogen or an organic residue, characterized in that the linking group T in total contains 2-500 carbon atoms and forms, together with the carbon atoms it is directly connected to it and the nitrogen atom, a substituted, 5-, 6 or 7-membered cyclic ring structure, or wherein R₂₄ and R₂₅ together are ═O or wherein R₂₄ is hydrogen and R₂₅ is hydrogen or hydroxy. T is most preferably 2-hydroxy-1,3-propanediyl or 2-oxo-1,3-propanediyl.
Preferred products of the formula (I) are those wherein G₁, G₂, G₃ and G₄, independently of each other, are methyl, ethyl, phenyl or COOR₄;
E is a carbon centered radical formed from a C₇-C₁₁phenylalkane or a C₆-C₁₀pyridylalkane; or C₅-C₁₂cycloalkane; or C₅-C₁₂cycloalkene; or an oxacyclohexane or oxycyclohexene; or C₃-C₈alkene; or C₃-C₈alkene substituted by phenoxy; or a benzene which is substituted by C₁-C₄alkyl and a further substituent selected from C₁-C₄alkoxy, glycidyl or glycidyloxy; or E is a radical of formula (VIII) 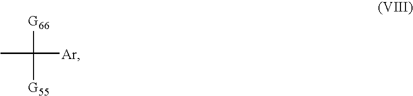
   wherein
Ar is C₆-C₁₀aryl or C₅-C₉heteroaryl;
G₁₄ is C₁-C₄alkyl or an acyl radical of an aliphatic carboxylic acid containing 2 to 4 carbon atoms or benzoyl;
G₅₅ is H, CH₃ or phenyl;
G₆₆ is —CN or a group of the formula —COOR₄ or —CH₂—O-G₁₄;
R₄ is hydrogen or C₁-C₈alkyl;
L is a carbon centered radical formed from propane, butane, pentane, 2,2-dimethyl-propane, xylene; and
T is phenylene or an organic linking group of the formula 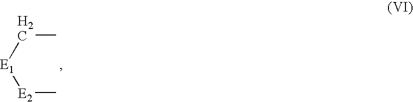
   wherein
E₂ is —CO— or —(CH₂)_{b}—, while b is 0, 1 or 2;
E₁ is a carbon atom carrying the two residues R₂₄ and R₂₅, or is >N—R₂₅, or is oxygen, and R₂₄ and R₂₅ are hydrogen or an organic residue, characterized in that the linking group T in total contains 2-500 carbon atoms and forms, together with the carbon atoms it is directly connected to it and the nitrogen atom, a substituted, 5-, 6 or 7-membered cyclic ring structure, or wherein R₂₄ and R₂₅ together are ═O or wherein R₂₄ is hydrogen and R₂₅ is hydrogen or hydroxy;
or E₁ and E₂ together are 1,2-phenylene.

The product of formula A most preferably corresponds to one of the formulae 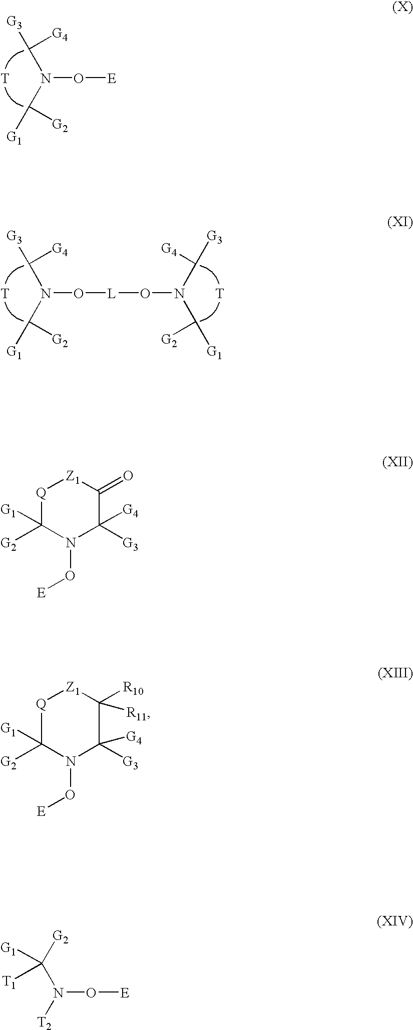
wherein
G₁, G₂, G₃ and G₄ independently of each other are C₁-C₁₈alkyl; C₃-C₁₈alkenyl; C₃-C₁₈alkinyl; C₁-C₁₈alkyl or C₃-C₁₈alkenyl or C₃-C₁₈alkinyl substituted by OH, halogen or a group —O—C(O)—R₅; C₂-C₁₈alkyl which is interrupted by O; C₅-C₁₂cycloalkyl; or phenyl; or G₁ and G₂ and/or G₃ and G₄ together with the linking carbon atom form a C₅-C₁₂cycloalkyl radical;
Z, is O or NR₈;
R₈ is hydrogen, OH, C₁-C₁₈alkyl, C₃-C₁₈alkenyl, C₃-C₁₈alkinyl, C₁-C₁₈alkyl, C₃-C₁₈alkenyl, C₃-C₁₈alkinyl which are substituted by one or more OH, halogen or a group —O—C(O)—N, C₂-C₁₈alkyl which is interrupted by at least one O atom and/or NR₅ group, C₃-C₁₂cycloalkyl or C₆-C₁₀aryl, C₇-C₉phenylalkyl, C₅-C₁₀heteroaryl, —C(O)—C₁-C₁₈alkyl, —O—C₁-C₁₈alkyl or —COOC₁-C₁₈alkyl;
Q is a direct bond or a divalent radical CR₉R₁₀, CR₉R₁₀—CR₁₁R₁₂, CR₉R₁₀CR₁₁R₁₂CR₁₃R₁₄, C(O) or CR₉R₁₀C(O);
R₉, R₁₀, R₁₁, R₁₂, R₁₃ and R₁₄ are independently hydrogen, phenyl, or C₁-C₁₈alkyl;
T is CH₂—C(R₂₄)(R₂₅)—CH₂, wherein R₂₄ and R₂₅ together are ═O or independently are H, OH or an organic residue, characterized in that the linking group T in total contains 2-500 carbon atoms and optionally 1-200 hetero atoms selected from, oxygen, phosphorus, sulfur, silicon, halogen and tertiary nitrogen.

The sterically hindered aminoxides, also referred to as N-oxyl educts for the instant process which include compounds of formulae B, III or IIIa, are largely known in the art; they may be prepared by oxidation of the corresponding N—H hindered amine with a suitable oxygen donor, e.g. by the reaction of the corresponding N—H hindered amine with hydrogen peroxide and sodium tungstate as described by E. G. Rozantsev et al., in Synthesis, 1971, 192; or with tert-butyl hydroperoxide and molybdenum (VI) as taught in U.S. Pat. No. 4,691,015, or obtained in analogous manner.

The preferred amount of hydrocarbon for the instant process depends to some extent on the relative number of reactive hydrogens on the hydrocarbon reactant and the hindered amine nitroxyl compound. The reaction is typically carried out with a ratio of 1 to 100 moles of hydrocarbon per mole of nitroxyl moiety with the preferred ratio being 1 to 50 moles per mole of nitroxyl moiety, and the most preferred ratio being 1 to 30 moles of hydrocarbon per mole of nitroxyl moiety.

The preferred amount of organic hydroperoxide is 1 to 20 moles per mole of nitroxyl moiety, with the more preferred amount being 1 to 5 moles of peroxide per mole of nitroxyl moiety and the most preferred amount being 1 to 3 moles of peroxide per mole of nitroxyl moiety.

The organic hydroperoxide used in the process of present invention can be of the formula R—OOH, wherein R usually is a hydrocarbon containing 1-18 carbon atoms. The organic hydroperoxide preferably is a peroxoalcohol containing 3-18 carbon atoms. R is often aliphatic, preferably C₁-C₁₂alkyl. Most preferred organic hydroperoxide is tert.butyl hydroperoxide.

The preferred amount of iodide catalyst is from about 0.0001 to 0.5, especially 0.0005 to 0.1 molar equivalent per mole of nitroxyl moiety, with a ratio of 0.001 to 0.05 moles of iodide per mole of nitroxyl moiety being the most preferred.

The reaction is preferably run at 0° to 100° C.; more preferably at 20° to 100° C., especially in the range 20-80° C.

More specifically, the instant process involves the reaction of a mixture of 1 to 100 moles of the hydrocarbon, e.g. of formula IV or V, 1 to 20 moles of organic hydroperoxide, and 0.001 mmoles to 0.5 moles of iodide catalyst per mole of N-oxyl compound, such as the compound of formula B (1 mmol is 0.001 mol). Preferably, the molar ratio of iodide catalyst per mole of N-oxyl compound is in the range from 1:100 to 1:100000, especially 1:300 to 1:100000.

E is preferably a carbon centered radical formed from a C₇-C₁₁phenylalkane or a C₆-C₁₀pyridylalkane; or C₅-C₁₂cycloalkane; or C₅-C₁₂cycloalkene; or an oxacyclohexane or oxycyclohexene; or C₃-C₈alkene; or C₃-C₈alkene substituted by phenoxy; or a benzene which is substituted by C₁-C₄alkyl and a further substituent selected from C₁-C₄alkoxy, glycidyl or glycidyloxy; or E is a radical of formula (VIII) 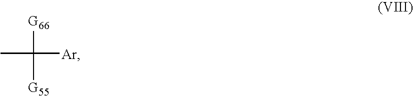
wherein
Ar is C₆-C₁₀aryl or C₅-C₉heteroaryl;
G₁₄ is C₁-C₄alkyl or an acyl radical of an aliphatic carboxylic acid containing 2 to 4 carbon atoms or benzoyl;
G₅₅ is H, CH₃ or phenyl;
G₆₆ is —CN or a group of the formula —COOR₄ or —CH₂—O-G₁₄;
R₄ is hydrogen or C₁-C₈alkyl;
L is a carbon centered radical formed from propane, butane, pentane, 2,2-dimethyl-propane, xylene.

Important are those educts, which are pure hydrocarbons.

The educt hydrocarbon, such as compound of formula IV or V, may serve two functions both as reactant and as solvent for the reaction. The reaction can also be carried out using an inert organic or inorganic solvent. A mixture of products may result if the hydrocarbon contains non-equivalent carbon-hydrogen bonds which are reactive in the instant process. For example, cyclohexane can give only one product whereas isopentane can give three distinct reaction products.

Usually the hydrocarbon reactand, e.g. compound of formula IV or V, reacts with its most active aliphatic carbon-hydrogen bond.

A solvent may be used, especially if the hydrocarbon, such as the compound of of formula IV or V, is a solid at the temperature of the reaction or if the catalyst is not very soluble in the hydrocarbon. Inert solvents should have less active carbon-hydrogen bonds; typical inert solvents are acetonitrile, aromatic hydrocarbons like benzene, chlorobenzene, CCl₄, alcohols (e.g. methanol, ethanol, ethylene glycol, ethylene glycol monomethyl ether), or, especially for reactions with activated hydrocarbons like alkylated aromats or alkenes, also alkanes like hexane, decane etc., or mixtures thereof. Inorganic solvents such as water are possible as well. The reaction can be carried out in one liquid phase or in separate phases.

Good results can be achieved when phase transfer catalysts such as quaternary ammonium or phosphonium salts are used. For example, quaternary ammonium or phosphonium halogenides such as chlorides or bromides may be employed for this purpose. The structure of the ammonium or phosphonium cation is less important; usually, quaternary ammonium or phosphonium cations contain 4 hydrocarbon residues bonded to the central nitrogen or phosphorus atom, which may be, for example, alkyl, phenylalkyl or phenyl groups. Some readily available materials are tetra-C₁-C₁₂alkylated.

The iodide catalyst may be selected from any iodide compound, including organic and inorganic iodide compounds. Examples are alkaline or alkaline earth metal iodides, or onium iodides such as ammonium or phosphonium or sulfonium iodides. Suitable metal iodides are, inter alia, those of lithium, sodium, potassium, magnesium or calcium.

Especially good results can be achieved when onium iodides are used which are soluble in organic solvents. Suitable onium iodides embrace quaternary ammonium, phosphonium or sulfonium iodides. The structure of the onium cation is less important provided the solubility in organic solvents is high enough; the latter can be increased by increasing the hydrophobicity of the hydrocarbon residues attached to the onium cation. Some readily available materials are tetra-C₁-C₁₂alkylated ammonium iodides and/or the following compounds:
Tetrabutylammonium iodide;
Tetraoctylammonium iodide;
Tetra(hexadecyl)ammonium iodide;
Tetradodecylammonium iodide;
Tetrahexylammonium iodide;
Di-octadecyl-dimethyl-ammonium iodide;
Hexadecyl-benzyl-dimethyl-ammonium iodide;
Tributyl-methyl-ammonium iodide^{A)};
Di-tetradecyl-dimethyl-ammonium iodide;
Trioctyl-propyl-ammonium iodide;
Octyl-benzyl-dimethyl ammonium iodide;
Trioctylmethylammonium iodide^{B)};
Hexadecylpyridinium iodide;
Dioctyl-dimethyl-ammonium iodide;
Octyl-trimethylammonium iodide;
Tetraethyl ammonium iodide;
Dioctyl-methyl sulfonium iodide;
Tetraphenylphosphonium iodide;
Triphenyl-isopropyl phosphonium iodide;
Triphenylethylphosphonium iodide;
Triphenylhexylphosphonium iodide;
Tetrabutyl phosphonium iodide;
Tributyl-hexadecyl phosphonium iodide;
Tetraoctyl phosphonium iodide;
Triphenylmethyl phosphonium iodide;
Diphenyl-dimethyl-phosphonium iodide;
Tetraethylphosphonium iodide;
Phenyl-trimethyl-phosphonium iodide;
Triphenyl-(CH₂CO₂CH₃)phosphonium iodide;
Triphenylbenzylphosphonium iodide.
A) iodide form of ALIQUAT® 175
B) iodide form of ALIQUAT® 336

In a preferred embodiment, the iodide catalyst functions the same time as a phase transfer catalyst, e.g. when a quaternary ammonium or phosphonium iodide such as tetrabutylammoniumiodide is used as catalyst. These compounds are known, many are commercially available.

The onium iodides can be generated from any other onium salt (e.g., hydroxide, sulfate, hydrogensulfate, fluoride, acetate, chloride, cyanide, bromide, nitrate, nitrite, perchlorate etc.) via insitu anion exchange using a watersoluble inorganic iodide such as alkaline or alkaline earth metal iodides, other iodine containing salts or elemental iodine. For example, commercial onium chlorides of the ALIQUAT® series may conveniently be brought into the above iodide form by in situ anion exchange.

The onium iodides can be bound to an organic or inorganic polymer backbone, rendering a homogeneous or heterogenous catalytic system.

Preferably, the pH of the aqueous phase, if present, is held between 7 and 11, especially between 9 and 10, most preferably at 9 during the reaction.

Preferred are quaternary ammonium or phosphonium iodides, especially tetraalkyl ammonium iodides.

The instant process can be run in air or in an inert atmosphere such a nitrogen or argon. The instant process can be run under atmospheric pressure as well as under reduced or elevated pressure. Elevated pressure can especially be useful in reactions with a hydrocarbon, which is gaseous under atmospheric pressure and the reaction temperature; in this case, pressure/temperature conditions are advantageous where the hydrocarbon forms a liquid phase or is at least partially dissolved in a suitable solvent.

There are several variations of the instant process. One variation involves the addition of a solution of organic hydroperoxide to a mixture of the N-oxyl hindered amine, the hydrocarbon and cosolvent (if used), and catalyst which has been brought to the desired temperature for reaction. The proper temperature may be maintained by controlling the rate of peroxide addition and/or by using a heating or cooling bath. After the hydroperoxide is added, the reaction mixture is conveniently stirred till the starting N-oxyl, e.g. compound of formula III, has disappeared or is no longer being converted to the desired product, e.g. compound of formula I and/or II. The reaction can be monitored by methods known in the art such as UV-Vis spectroscopy, thin layer chromatography, gas chromatography or liquid chromatography. Additional portions of catalyst can be added while the reaction is in progress. After the initial hydroperoxide charge has been added to the reaction mixture, more hydroperoxide can be added dropwise to bring the reaction to completion.

A second variation of the instant process is to simultaneously add separate solutions of the hydroperoxide and the nitroxyl compound to a mixture of the hydrocarbon, cosolvent (if used) and catalyst. The nitroxyl compound may be dissolved in water or the alcohol solvent used in the reaction. Some of the nitroxyl compound may be introduced into the reaction mixture prior to starting the peroxide addition, and all of the nitroxyl compound should be added prior to completing the peroxide addition.

Another variation of the instant process involves the simultaneous addition of separate solutions of the hydroperoxide and of the aqueous or alcohol solution of the catalyst to a mixture of the nitroxyl compound, hydrocarbon, and cosolvent (if used). Some of the metal may be introduced into the reaction mixture prior to starting the peroxide addition.

Still another variation of the instant process is the simultaneous addition of separate solutions of the hydroperoxide, of the aqueous or alcohol solution of the nitroxyl compound, and of an aqueous or alcohol solution of the catalyst to the hydrocarbon and cosolvent (if used). A portion of the nitroxyl compound and/or catalyst may be introduced into the reaction mixture prior to starting the hydroperoxide addition. All of the nitroxyl compound should be added prior to completing the hydroperoxide addition.

At the end of the reaction, the residual hydroperoxide should be carefully decomposed prior to the isolation of any products.

Examples for compounds which can be obtained advantageously with the process of present invention are those of formulae 1-28:

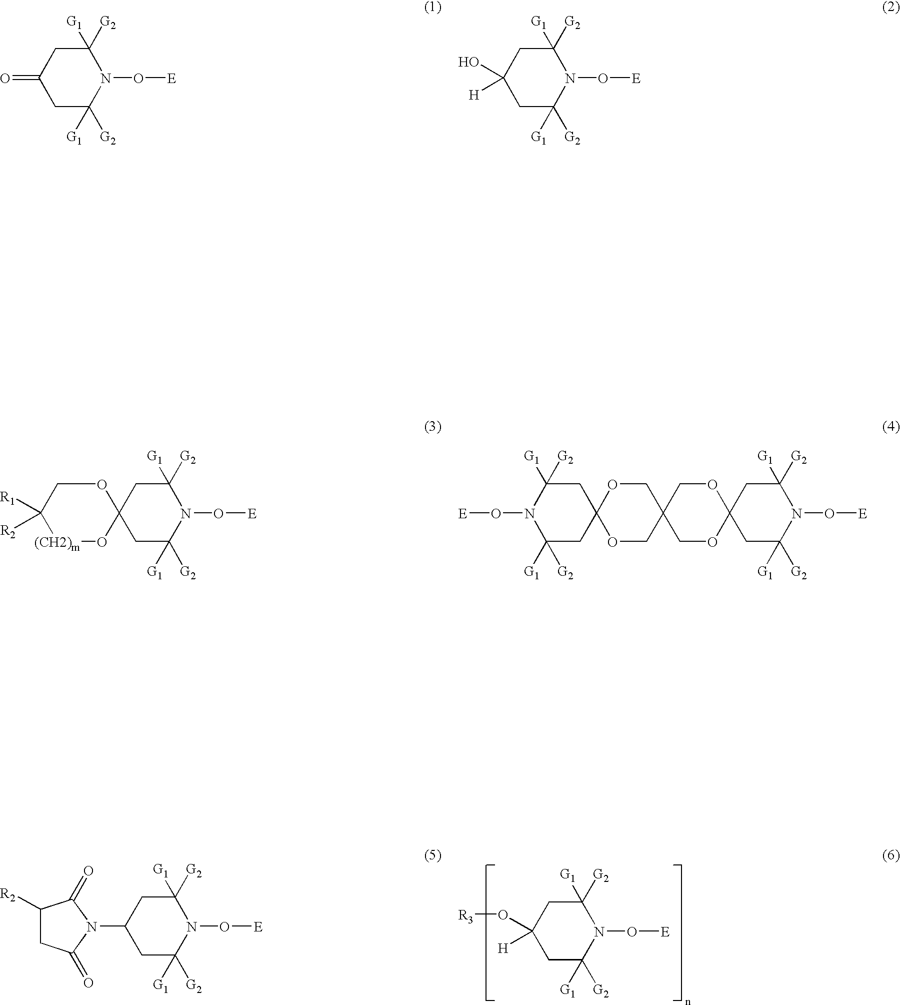

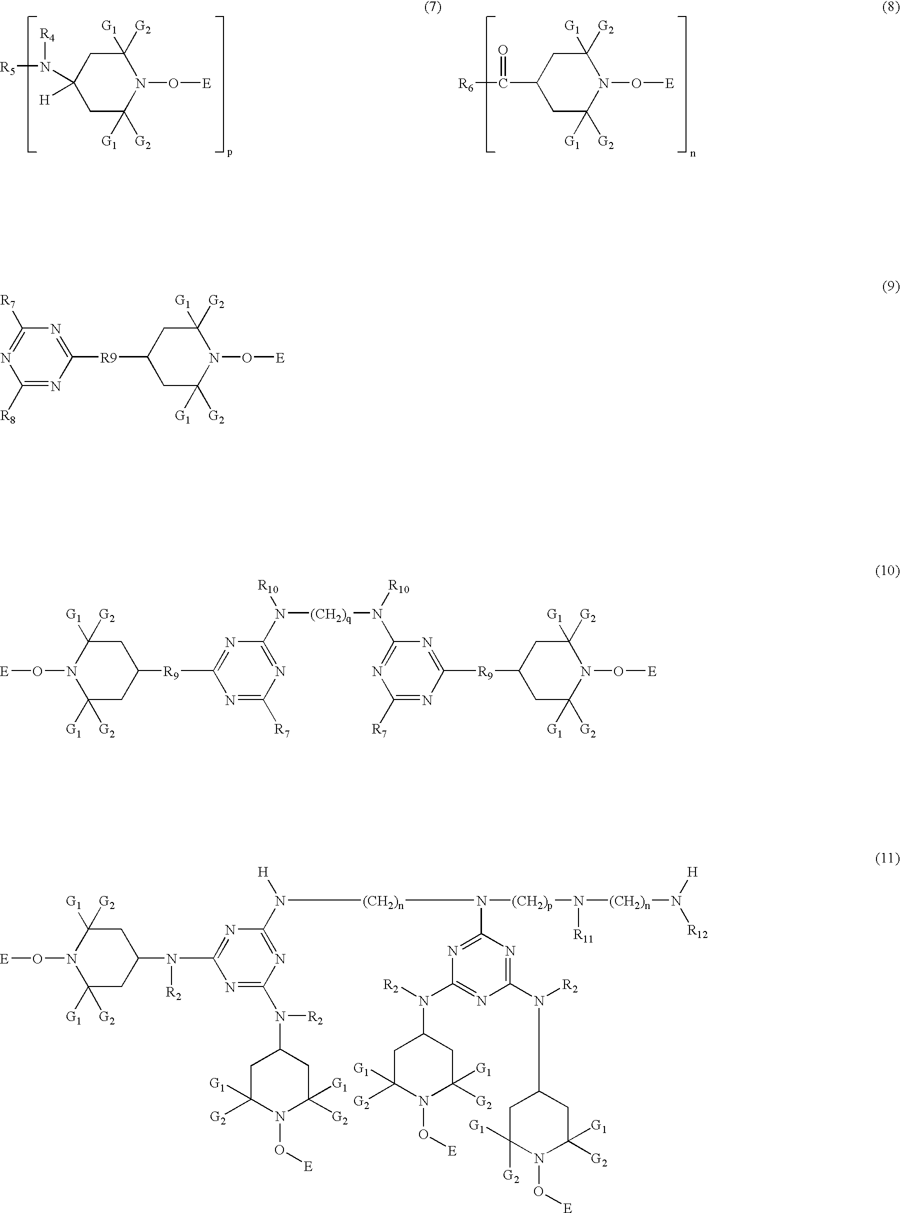

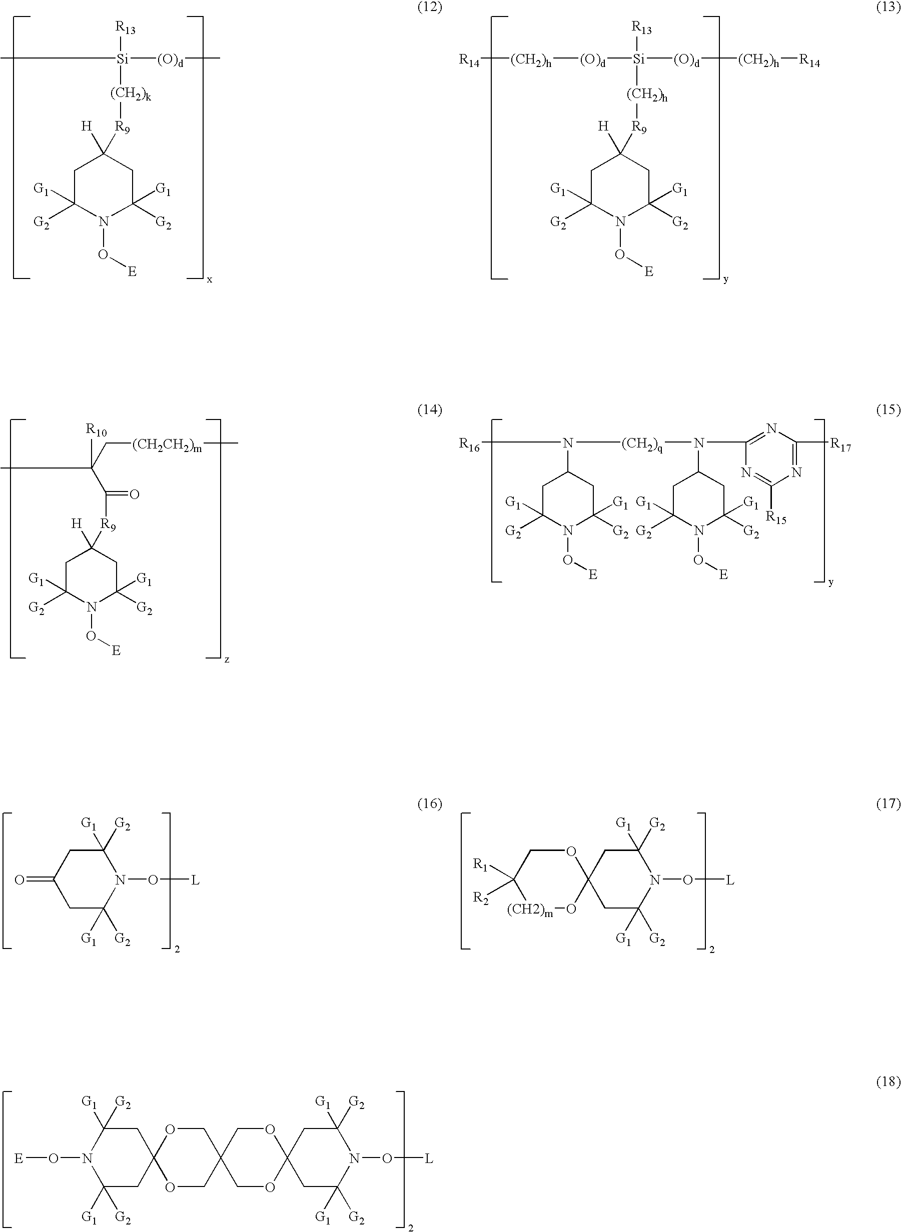

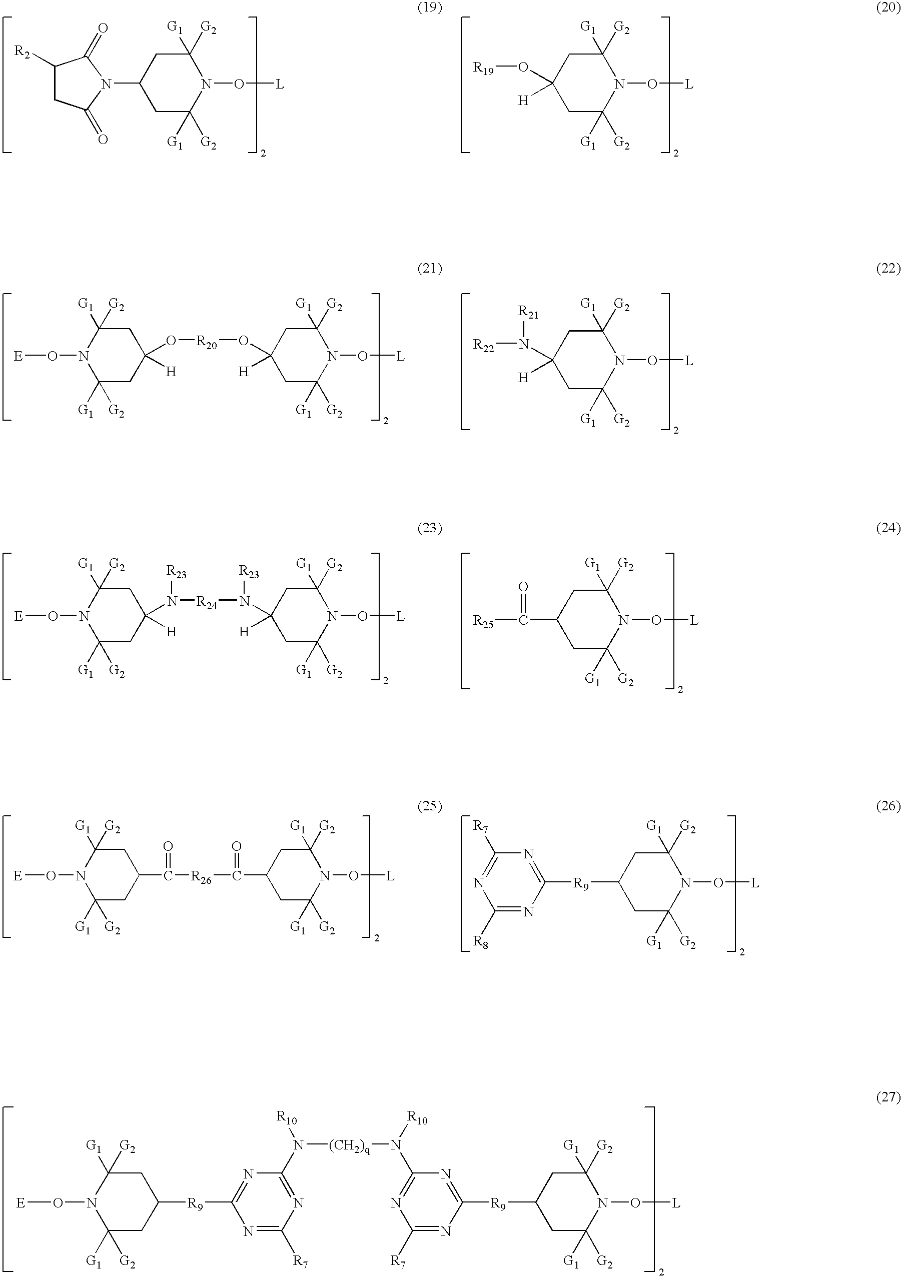

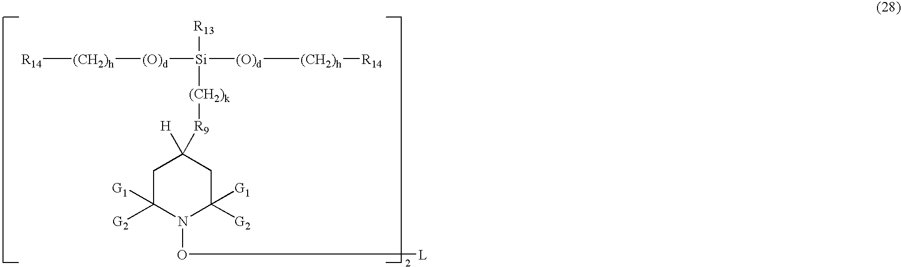

wherein in formulas (1) to (15):
m is 0 or 1;
R₁ is hydrogen, hydroxyl or hydroxymethyl;
R₂ is hydrogen, alkyl of 1 to 12 carbon atoms or alkenyl of 2 to 12 carbon atoms;
n is 1 to 4;
when n is 1,
R₃ is alkyl of 1 to 18 carbon atoms, alkoxycarbonylalkylenecarbonyl of 4 to 18 carbon atoms, alkenyl of 2 to 18 carbon atoms, glycidyl, 2,3-dihydroxypropyl, 2-hydroxy or 2-(hydroxymethyl) substituted alkyl of 3 to 12 carbon atoms which alkyl is interrupted by oxygen, an acyl radical of an aliphatic or unsaturated aliphatic carboxylic or carbamic acid containing 2 to 18 carbon atoms, an acyl radical of a cycloaliphatic carboxylic or carbamic acid containing 7 to 12 carbon atoms, or acyl radical of an aromatic acid containing 7 to 15 carbon atoms;
when n is 2,
R₃ is alkylene of 2 to 18 carbon atoms, a divalent acyl radical of an aliphatic or unsaturated aliphatic dicarboxylic or dicarbamic acid containing 2 to 18 carbon atoms, a divalent acyl radical of a cycloaliphatic dicarboxylic or dicarbamic acid containing 7 to 12 carbon atoms, or a divalent acyl radical of an aromatic dicarboxylic acid containing 8 to 15 carbon atoms;
when n is 3,
R₃ is a trivalent acyl radical of an aliphatic or unsaturated aliphatic tricarboxylic acid containing 6 to 18 carbon atoms, or a trivalent acyl radical of an aromatic tricarboxylic acid containing 9 to 15 carbon atoms;
when n is 4,
R₃ is a tetravalent acyl radical of an aliphatic or unsaturated aliphatic tetracarboxylic acid, especially 1,2,3,4-butanetetracarboxylic acid, 1,2,3,4-but-2-enetetracarboxylic acid, 1,2,3,5-pentanetetracarboxylic acid and 1,2,4,5-pentanetetracarboxylic acid, or R₃ is a tetravalent acyl radical of an aromatic tetracarboxylic acid containing 10 to 18 carbon atoms;
p is 1 to 3,
R₄ is hydrogen, alkyl of 1 to 18 carbon atoms or acyl of 2 to 6 carbon atoms;
when p is 1,
R₅ is hydrogen, alkyl of 1 to 18 carbon atoms, an acyl radical of an aliphatic or unsaturated aliphatic carboxylic or carbamic acid containing 2 to 18 carbon atoms; an acyl radical of a cycloaliphatic carboxylic or carbamic acid containing 7 to 12 carbon atoms, an acyl radical of an aromatic carboxylic acid containing 7 to 15 carbon atoms, or R₄ and R₅ together are —(CH₂)₅CO—, phthaloyl or a divalent acyl radical of maleic acid;
when p is 2,
R₅ is alkylene of 2 to 12 carbon atoms, a divalent acyl radical of an aliphatic or unsaturated aliphatic dicarboxylic or dicarbamic acid containing 2 to 18 carbon atoms, a divalent acyl radical of a cycloaliphatic dicarboxylic or dicarbamic acid containing 7 to 12 carbon atoms, or a divalent acyl radical of an aromatic dicarboxylic acid containing 8 to 15 carbon atoms;
when p is 3,
R₅ is a trivalent acyl radical of an aliphatic or unsaturated aliphatic tricarboxylic acid containing 6 to 18 carbon atoms, or a trivalent acyl radical of an aromatic tricarboxylic acid containing 9 to 15 carbon atoms;
when n is 1,
R₆ is alkoxy of 1 to 18 carbon atoms, alkenyloxy of 2 to 18 carbon atoms, —NHalkyl of 1 to 18 carbon atoms or —N(alkyl)₂ of 2 to 36 carbon atoms,
when n is 2,
R₆ is alkylenedioxy of 2 to 18 carbon atoms, alkenylenedioxy of 2 to 18 carbon atoms, —NH-alkylene-NH— of 2 to 18 carbon atoms or —N(alkyl)-alkylene-N(alkyl)- of 2 to 18 carbon atoms, or R₆ is 4-methyl-1,3-phenylenediamino,
when n is 3,
R₆ is a trivalent alkoxy radical of a saturated or unsaturated aliphatic triol containing 3 to 18 carbon atoms,
when n is 4,
R₆ is a tetravalent alkoxy radical of a saturated or unsaturated aliphatic tetraol containing 4 to 18 carbon atoms,
R₇ and R₈ are independently chlorine, alkoxy of 1 to 18 carbon atoms, —O-T₁, amino substituted by 2-hydroxyethyl, —NH(alkyl) of 1 to 18 carbon atoms, —N(alkyl)T₁ with alkyl of 1 to 18 carbon atoms, or —N(alkyl)₂ of 2 to 36 carbon atoms,
R₉ is oxygen, or R₉ is nitrogen substituted by either hydrogen, alkyl of 1 to 12 carbon atoms or T₁
   T₁ is 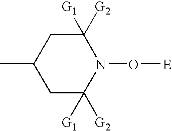
R₁₀ is hydrogen or methyl,
q is 2 to 8,
R₁₁ and R₁₂ are independently hydrogen or the group T₂ 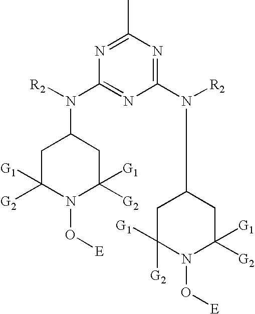
R₁₃ is hydrogen, phenyl, straight or branched alkyl of 1 to 12 carbon atoms, alkoxy of 1 to 12 carbon atoms, straight or branched alkyl of 1 to 4 carbon atoms substituted by phenyl, cycloalkyl of 5 to 8 carbon atoms, cycloalkenyl of 5 to 8 carbon atoms, alkenyl of 2 to 12 carbon atoms, glycidyl, allyloxy, straight or branched hydroxyalkyl of 1 to 4 carbon atoms, or silyl or silyloxy substituted three times independently by hydrogen, by phenyl, by alkyl of 1 to 4 carbon atoms or by alkoxy of 1 to 4 carbon atoms;
R₁₄ is hydrogen or silyl substituted three times independently by hydrogen, by phenyl, by alkyl of 1 to 4 carbon atoms or by alkoxy of 1 to 4 carbon atoms;
d is 0 or 1;
h is 0 to 4;
k is 0 to 5;
x is 3 to 6;
y is 1 to 10;
z is an integer such that the compound has a molecular weight of 1000 to 4000 amu, e.g. z may be from the range 3-10;
R₁₅ is morpholino, piperidino, 1-piperizinyl, alkylamino of 1 to 8 carbon atoms, especially branched alkylamino of 3 to 8 carbon atoms such as tert-octylamino, —N(alkyl)T₁ with alkyl of 1 to 8 carbon atoms, or —N(alkyl)₂ of 2 to 16 carbon atoms,
R₁₆ is hydrogen, acyl of 2 to 4 carbon atoms, carbamoyl substituted by alkyl of 1 to 4 carbon atoms, s-triazinyl substituted once by chlorine and once by R₁₅, or s-triazinyl substituted twice by R₁₅ with the condition that the two R₁₅ substituents may be different;
R₁₇ is chlorine, amino substituted by alkyl of 1 to 8 carbon atoms or by T₁, —N(alkyl)T₁ with alkyl of 1 to 8 carbon atoms, —N(alkyl)₂ of 2 to 16 carbon atoms, or the group T₃ 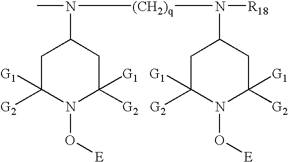
R₁₈ is hydrogen, acyl of 2 to 4 carbon atoms, carbamoyl substituted by alkyl of 1 to 4 carbon atoms, s-triazinyl substituted twice by —N(alkyl)₂ of 2 to 16 carbon atoms or s-triazinyl substituted twice by —N(alkyl)T₁ with alkyl of 1 to 8 carbon atoms;
in formulas (16) to (28), R₁, R₂, R₇, R₈, R₉, R₁₀, R₁₃, R₁₄, d, h, k, m, q, and T₁ have the same meanings as in formulas (1) to (15);
R₁₉ is hydrogen, alkyl of 1 to 18 carbon atoms, alkenyl of 2 to 18 carbon atoms, glycidyl, 2,3-dihydroxypropyl, 2-hydroxy or 2-(hydroxymethyl) substituted alkyl of 3 to 12 carbon atoms which alkyl is interrupted by oxygen, an acyl radical of an aliphatic or unsaturated aliphatic carboxylic or carbamic acid containing 2 to 18 carbon atoms, an acyl radical of a cycloaliphatic carboxylic or carbamic acid containing 7 to 12 carbon atoms, or acyl radical of an aromatic acid containing 7 to 15 carbon atoms;
R₂₀ is alkylene of 2 to 18 carbon atoms, a divalent acyl radical of an aliphatic or unsaturated aliphatic dicarboxylic or dicarbamic acid containing 2 to 18 carbon atoms, a divalent acyl radical of a cycloaliphatic dicarboxylic or dicarbamic acid containing 7 to 12 carbon atoms, or a divalent acyl radical of an aromatic dicarboxylic acid containing 8 to 15 carbon atoms;
R₂₁ is hydrogen, alkyl of 1 to 18 carbon atoms or acyl of 2 to 6 carbon atoms;
R₂₂ is hydrogen, alkyl of 1 to 18 carbon atoms, an acyl radical of an aliphatic or unsaturated aliphatic carboxylic or carbamic acid containing 2 to 18 carbon atoms, an acyl radical of a cycloaliphatic carboxylic or carbamic acid containing 7 to 12 carbon atoms, an acyl radical of an aromatic carboxylic acid containing 7 to 15 carbon atoms, or R₄ and R₅ together are —(CH₂)₅CO—, phthaloyl or a divalent acyl radical of maleic acid;
R₂₃ is hydrogen, alkyl of 1 to 4 carbon atoms or acyl of 2 to 6 carbon atoms;
R₂₄ is alkylene of 2 to 18 carbon atoms, a divalent acyl radical of an aliphatic or unsaturated aliphatic dicarboxylic or dicarbamic acid containing 2 to 18 carbon atoms, a divalent acyl radical of a cycloaliphatic dicarboxylic or dicarbamic acid containing 7 to 12 carbon atoms, or a divalent acyl radical of an aromatic dicarboxylic acid containing 8 to 15 carbon atoms;
R₂₅ is alkoxy of 1 to 18 carbon atoms, alkenyloxy of 2 to 18 carbon atoms, —NHalkyl of 1 to 18 carbon atoms or —N(alkyl)₂ of 2 to 36 carbon atoms,
R₂₆ is alkylenedioxy of 2 to 18 carbon atoms, alkenylenedioxy of 2 to 18 carbon atoms, —NH-alkylene-NH— of 2 to 18 carbon atoms or —N(alkyl)-alkylene-N(alkyl)- of 3 to 18 carbon atoms.
   E is a carbon centered radical formed preferably from a C₇-C₁₁phenylalkane, especially toluene, ethylbenzene, isopropylbenzene; or C₅-C₁₂cycloalkane, especially cyclohexene; or C₅-C₁₂cycloalkene, especially cyclohexene; or C₃-C₈alkene, especially propene; or a benzene which is substituted by C₁-C₄alkyl and a further substituent selected from C₁-C₄alkoxy, glycidyl or glycidyloxy.
   L is a carbon centered radical formed preferably from propane, butane, pentane, 2,2-dimethyl-propane, xylene, diethylbenzene.

Preferably, the reaction site in the compound E-H or H-L-H is an activated carbon-hydrogen bond, whose carbon, for example, is linked to an electron pushing functional group or a functional group able to stabilize the radical formed after cleavage of the carbon-hydrogen bond. Electron withdrawing groups, if present in E-H or H-L-H, are preferably not directly linked to the reactive site.

Products of the present process can be employed with advantage for stabilizing organic material against the damaging effect of light, oxygen and/or heat, especially for stabilizing synthetic organic polymers or compositions containing them. They are notable for high thermal stability, substrate compatibility and good persistence in the substrate.

The compounds made by the instant process are particularly effective in the stabilization of polymer compositions against harmful effects of light, oxygen and/or heat; they are also useful as initiators or regulators for radical polymerization processes which provide homopolymers, random copolymers, block copolymers, multiblock copolymers, graft copolymers and the like, at enhanced rates of polymerization and enhanced monomer to polymer conversions.

Of particular interest is the use of products of the present process as stabilizers in synthetic organic polymers, for example a coating or a bulk polymer or article formed therefrom, especially in thermoplastic polymers and corresponding compositions as well as in coating compositions. Thermoplastic polymers of most importance in present compositions are polyolefines and their copolymers, thermoplastic polyolefin (TPO), thermoplastic polyurethan (TPU), thermoplastic rubber (TPR), polycarbonate, such as in item 19 above, and blends, such as in item 28 above. Of utmost importance are polyethylene (PE), polypropylene (PP), polycarbonate (PC) and polycarbonate blends such as PC/ABS blends, as well as in acid or metal catalyzed coating compositions.

In general the products of present invention may be added to the material to be stabilized in amounts of from 0.1 to 10%, preferably from 0.01 to 5%, in particular from 0.01 to 2% (based on the material to be stabilized). Particular preference is given to the use of the novel compounds in amounts of from 0.05 to 1.5%, especially from 0.1 to 0.5%. Where compounds of present invention are used as flame retardants, dosages are usually higher, e.g. 0.1 to 25% by weight, mainly 0.1 to 10% by weight of the organic material to be stabilized and protected against inflammation.

Used in polymerizable compositions as a polymerization regulator or initiator, preferably the regulator/initiator compound is present in an amount of from 0.01 mol-% to 30 mol-%, more preferably in an amount of from 0.1 mol-% to 20 mol-% and most preferred in an amount of from 0.5 mol-% to 10 mol-% based on the monomer or monomer mixture.

The following examples are for illustrative purposes only and are not to be construed to limit the instant invention in any manner whatsoever. Percentages given are usually percent by weight if not otherwise indicated. Abbreviations used:
min. minutes;
HPLC high pressure liquid chromatography;
GC gas chromatography;
Bu butyl;
Ph phenyl;
Me methyl;
Oct octyl;
Hex hexyl;
Et ethyl;
Bz benzyl;
Py 1-pyridinium;
TEMPO 2,2,6,6-tetramethylpiperidine-N-oxide;
eq. equivalent (of nitroxide, if not otherwise indicated).

### EXAMPLE 1

### Preparation of the Compound of Formula

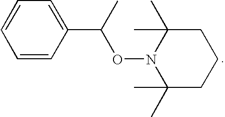

To a stirred mixture of 5 g (32 mmol) 2,2,6,6-tetramethylpiperidine-N-oxide (TEMPO), 34 g (320 mmol) of ethylbenzene and 0.12 g (0.32 mmol) of tetrabutylammoniumiodide, 6.2 g (48 mmol) of t-butylhydroperoxid (70% aqueous solution) are added at 60° C. within 30 minutes. The temperature is maintained at 60° C. for 25 minutes until all of the TEMPO has reacted. The reaction mixture is cooled down to 25° C. and stirred with 61 g of an aqueous solution of Na₂SO₃ (10%) until the disappearance of excess t-butylhydroperoxide. The aqueous phase is then separated and washed with ethylbenzene. The combined organic phases are washed with brine, dried over MgSO₄, filtered, and the solvent is distilled off on a rotary-evaporator. The crude product is purified by flash-chromatography (silica gel, hexane:ethylacetate 9:1), yielding 5 g (60% of theory) of a yellow oil. Analysis required for C₁₇H₂₇NO (261.41): C, 78.11%, H, 10.41%, N, 5.36%; found: C, 78.04%, H, 10.46%, N, 5.26%. ¹H-NMR (CDCl₃), δ (ppm): 0.66 (broad s, 3H), 1.03-1.52 (m, 15H), 1.48 (d, J=8 Hz, 3H), 4.78 (q, J=8 Hz, 1H), 7.21-7.33 (m, 5H).

### EXAMPLE 2

Example 1 is repeated except that 32 mmol of 2,2,6,6-Tetramethylpiperidine-N-oxide are replaced by the equivalent amount of 2,2,6,6-Tetramethylpiperidine-4-one-N-oxide, yielding a compound of formula

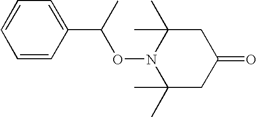

### EXAMPLE 3

### Preparation of a Compound of Formula

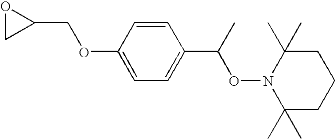

A stirred mixture of 0.5 g (3.2 mmol) TEMPO, 1.14 g (6.4 mmol) of 2-(4-ethyl-phenoxymethyl)-oxirane, 0.0118 g (0.032 mmol) of tetrabutylammoniumiodide and 0.62 g (4.8 mmol) of t-butylhydroperoxid (70% aqueous solution) is brought to 60° C. The temperature is maintained at 60° C. for 4 hours until all of the TEMPO has reacted. The reaction mixture is cooled down to 25° C. and stirred with 20 g of a 10% aqueous Na₂SO₃ solution until the disappearance of excess t-butylhydroperoxide. The aqueous phase is then separated and washed with ethylbenzene. The combined organic phases are passed through a plug of silica gel, washed with brine, dried over MgSO₄, filtered and the solvent distilled off on a rotary-evaporator, yielding 0.9 g of a colorless oil. Quantitative HPLC-analysis reveals a product-concentration of 65% w/w, corresponding to an overall yield of 54.8%. ¹H-NMR (CDCl₃), δ (ppm; 2-(4-Ethyl-phenoxymethyl)-oxirane not shown): 0.63 (broad s, 3H), 1.01-1.56 (m, 15H), 1.45 (d, J=8 Hz, 3H), 2.75-2.76 (m, 1H), 2.89-2.91 (m, 1H), 3.34-3.36 (m, 1H), 3.95-3.99 (m, 1H), 4.17-4.21 (m, 1H), 4.73 (q, J=8 Hz, 1H), 6.84-6.88 (m, 2H), 7.21-7.26 (m, 2H).

### EXAMPLE 4

### Preparation of the Compound of Formula

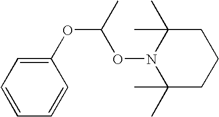

To a stirred mixture of 5 g (32 mmol) TEMPO, 39.1 g (320 mmol) of phenetole and 0.12 g (0.32 mmol) of tetrabutylammoniumiodide, 12.37 g (96 mmol) of t-butylhydroperoxid (70% aqueous solution) are added at 60° C. within 60 minutes. The temperature is maintained at 60° C. for 21 hours until all TEMPO has reacted. The reaction mixture is cooled down to 25° C. and stirred with 121 g of a 10% aqueous Na₂SO₃ solution until the disappearance of excess t-butylhydroperoxide. The aqueous phase is then separated and washed with cyclohexane. The combined organic phases are washed with brine, dried over MgSO₄, filtered. and the solvent is distilled off on a rotary-evaporator. The crude product is purified by flash-chromatography (silica gel, Hexane/Ethylacetate 9/1), yielding 4.6 g (51.8% of theory) of a slightly yellow oil. Analysis required for C₁₇H₂₇NO₂ (277.41): C, 73.61%, H, 9.81%, N, 5.05%; found: C, 73.15%, H, 9.89%, N, 4.95%. ¹H-NMR (CDCl₃), δ (ppm): 1.13 (s, 3H), 1.16 (s, 3H), 1.19 (s, 6H), 1.30-1.69 (m, 6H), 1.47 (d, J=8 Hz, 3H), 5.58 (q, J=8 Hz, 1H), 6.92-6.96 (m, 1H), 7.01-7.03 (m, 2H), 7.24-7.28 (m, 2H).

### EXAMPLE 5

### Preparation of

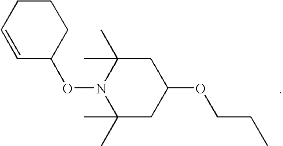

To a stirred mixture of 50 mmol 4-propoxy-2,2,6,6-tetramethylpiperidine-1-oxyl, 41.1 g (500 mmol) of cyclohexene and 0.18 g (0.5 mmol) of tetrabutylammoniumiodide, 7.4 g (58 mmol) of t-butylhydroperoxid (70% aqueous solution) are added at 55° C. within 30 minutes. The reaction mixture is cooled down to 25° C. and stirred with 63 g of an aqueous 20% Na₂SO₃ solution until the disappearance of excess t-butylhydroperoxide. The aqueous phase is then separated and washed with cyclohexane. The combined organic phases are passed through a plug of silica gel and washed with brine, dried over MgSO₄, filtered and the solvent distilled off on a rotary-evaporator. The crude product is purified by distillation, yielding the title product.

### EXAMPLE 6

### Preparation of

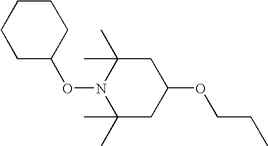 by Hydrogenation of the Product of Example 5

A mixture of 4 mmol) of the product of Example 5 and 0.2 g Pd on charcoal (10%) in 10 ml of methanol is hydrogenated at 25° C. and 4 bar of hydrogen. Filtration and evaporation of the solvent yields the title product as a slightly orange oil.

### EXAMPLE 7

### Preparation of the Compound of the Formula

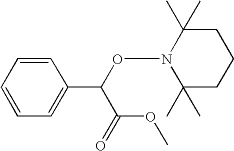

To a stirred mixture of 5.5 g (35 mmol) TEMPO, 10.5 g (70 mmol) of phenylacetic acid methyl ester and 0.13 g (0.35 mmol) of tetrabutylammoniumiodide, 6.75 g (52.5 mmol) of t-butylhydroperoxid (70% aqueous solution) are added at 60° C. within 25 minutes. The temperature is maintained at 60° C. for 46 hours. The reaction mixture is cooled down to 25° C. and stirred with 66 g of a 10% aqueous Na₂SO₃ solution until the disappearance of excess t-butylhydroperoxide. The aqueous phase is then separated and washed with ethylbenzene. The combined organic phases are washed with brine, dried over MgSO₄, filtered and the solvent distilled off on a rotary-evaporator. The crude product is purified by flash-chromatography (silica gel, hexane:ethylacetate 9:1), yielding 6 g (56% of theory) of the title product as a white crystalline solid, mp 85° C.-87° C. Analysis required for C₁₈H₂₇NO₃ (305.42): C, 70.79%, H, 8.91%, N, 4.59%; found: C, 70.60%, H, 9.13%, N, 4.53%. ¹H-NMR (CDCl₃), δ (ppm): 0.72 (s, 3H), 1.07 (s, 3H), 1.14 (s, 3H), 1.23 (s, 3H), 1.28-1.58 (m, 6H), 3.65 (s, 3H), 5.21 (s, 1H), 7.27-7.35 (m, 3H), 7.43-7.45 (d-like, 2H).

### EXAMPLE 8

### Preparation of the Compound of the Formula

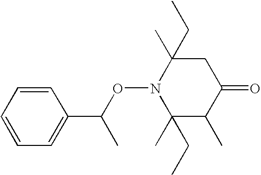

To a stirred mixture of 6.8 g (32 mmol) of 2,6-diethyl-2,3,6-trimethyl-piperidin-4-one-N-oxide, 34 g (320 mmol) of ethylbenzene and 0.12 g (0.32 mmol) of tetrabutylammoniumiodide, 6.2 g (48 mmol) of t-butylhydroperoxid (70% aqueous solution) are added at 60° C. within 30 minutes. The temperature is maintained at 60° C. for 13 hours, after which another 6.2 g of t-butylhydroperoxid and 0.12 g of tetrabutylammoniumiodide are added. The temperature is maintained at 60° C. for another 24 hours, cooled down to 25° C. and stirred with 120 g of a 10% aqueous Na₂SO₃ solution until the disappearance of excess t-butylhydroperoxide. The aqueous phase is then separated and washed with ethylbenzene. The combined organic phases are washed with brine, dried over MgSO₄, filtered and the solvent distilled off on a rotary-evaporator. The crude product is purified by flash-chromatography (silica gel, hexane:Ethylacetate 9:1), yielding the title product as a yellow oil. Analysis required for C₂₀H₃₁NO₂ (317.48): C, 75.67%, H, 9.84%, N, 4.41%; found: C, 74.01%, H, 9.76%, N, 4.30%. ¹H-NMR (CDCl₃), δ (ppm, O—CH only): 4.83 (p-like, 1H).

### EXAMPLE 9

### Preparation of the Compound of the Formula

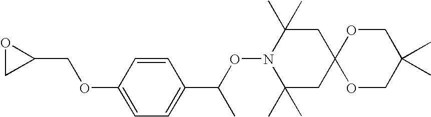

To a stirred mixture of 6.4 g (25 mmol) of 3,3,8,8,10,10-hexamethyl-1,5-dioxa-9-aza-spiro[5.5]undecane-N-oxide, 8.9 g (50 mmol) of 2-(4-ethyl-phenoxymethyl)-oxirane and 0.09 g (0.25 mmol) of tetrabutylammoniumiodide, 3.4 g (37.5 mmol) of t-butylhydroperoxid (70% aqueous solution) are added at 60° C. within 30 minutes. The temperature is maintained at 60° C. for 17.6 hours. The reaction mixture is cooled down to 25° C. and stirred with 47 g of an aqueous 10% Na₂SO₃ solution until the disappearance of excess t-butylhydroperoxide. The aqueous phase is then separated and washed with cyclohexane. The combined organic phases are washed with brine, dried over MgSO₄, filtered and the solvent distilled off on a rotary-evaporator, yielding 12.2 g of a brownish oil partially crystallizing at low temperature. The title product is obtained as an off-white solid, mp 106° C.-110° C. Analysis required for C₂₅H₃₉NO₅ (433.59): C, 69.25%, H, 9.07%, N, 3.23%; found: C, 68.24%, H, 9.04%, N, 2.87%. ¹H-NMR (CDCl₃), δ (ppm): 0.63 (br s, 3H), 0.93 (br s, 3H), 0.95 (br s, 3H), 1.14 (br s, 3H), 1.30 (br s, 6H), 1.45-1.48 (m, 4H), 1.53-1.60 (m, 1H), 2.05-2.09 (d-like, 1H), 2.16-2.20 (d-like, 1H), 2.75-2.76 (m, 1H), 2.89-2.91 (m, 1H), 3.34-3.36 (m, 1H), 3.45 (s, 4H), 3.94-3.99 (m, 1H), 4.18-4.21 (m, 1H), 4.74 (q, J=8 Hz, 1H), 6.84-6.87 (d-like, 2H), 7.22-7.25 (d-like, 2H).

### EXAMPLE 10

### Preparation of the Compound of the Formula

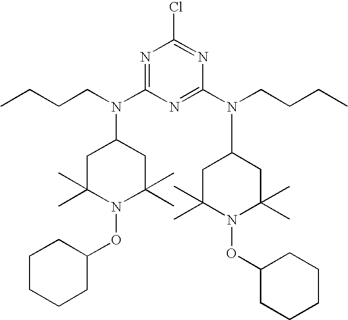

A stirred mixture of 1.42 g (2.5 mmol) of N,N′-dibutyl-6-chloro-N,N′-bis-(2,2,6,6-tetramethyl-piperidin-4-yl-N-oxide)-[1,3,5]-triazine-2,4-diamine, 4.2 g (50 mmol) cyclohexane, 0.018 g (0.05 mmol) tetrabutylammoniumiodide and 1.93 g (15 mmol) t-butylhydroperoxid (70% aqueous solution) is brought to 68° C. The temperature is maintained at 68° C. for 22 hours. The reaction mixture is cooled down to 25° C. and stirred with 18.9 g of an aqueous 10% Na₂SO₃ solution until the disappearance of excess t-butylhydroperoxide. The aqueous phase is then separated and washed with cyclohexane. The combined organic phases are washed with brine, dried over MgSO₄, filtered and the solvent distilled off on a rotary-evaporator, yielding 1.1 g g of a reddish solid. Purification by flash-chromatography (silica gel, hexane:ethylacetate 9:1) yields the title product as a white solid, mp 86° C.-90° C. Analysis required for C₄₁H₇₄ClN₇O₂ (732.55): C, 67.23%, H, 10.18%, Cl, 4.84%, N, 13.38%; found: C, 67.16%, H, 10.08%, Cl, 4.91%, N, 12.86%. ¹H-NMR (CDCl₃), δ (ppm): 0.88-0.96 (m, 6H), 1.05-1.4 (m, 42H), 1.45-1.60 (m, 6H), 1.63-1.80 (m, 8H), 2.0-2.1 (m, 4H), 3.25-3.35 (m, 4H), 3.55-3.65 (m, 2H), 4.9-5.1 (m, 2H).

### EXAMPLE 11

### Preparation of the Compound of the Formula

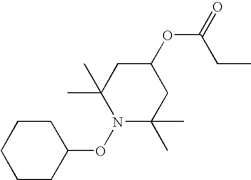

To a stirred mixture of 8 g (35 mmol) of propionic acid-2,2,6,6-tetramethylpiperidin-4-yl-N-oxide ester, 29.5 g (350 mmol) cyclohexane and 0.13 g (0.35 mmol) of tetrabutylammoniumiodide, 13.5 g (105 mmol) of t-butylhydroperoxid (70% aqueous solution) are added at 60° C. within 20 minutes. The temperature is maintained at 60° C. for 2.8 hours. The reaction mixture is cooled down to 25° C. and stirred with 132 g of an aqueous 10% Na₂SO₃ solution until the disappearance of excess t-butylhydroperoxide. The aqueous phase is then separated and washed with cyclohexane. The combined organic phases are washed with brine, dried over MgSO₄, filtered and the solvent distilled off on a rotary-evaporator, yielding 10 g of a reddish oil. Purification by flash-chromatography (silica gel, hexane:ethylacetate 9:1) yields the title product as a yellowish oil. Analysis required for C₁₈H₃₃NO₃ (311.47): C, 69.41%, H, 10.68%, N, 4.50%; found: C, 69.32%, H, 10.57%, N, 4.40%. ¹H-NMR (CDCl₃), δ (ppm): 1.09 (t, J=8 Hz, 3H), 1.10-1.26 (m, 17H), 1.52-1.57 (m, 3H), 1.74-1.84 (m, 4H), 2.03-2.05 (m, 2H), 2.28 (q, J=8 Hz, 2H), 3.56-3.62 (m, 1H), 4.98-5.06 (m, 1H).

### EXAMPLE 12

### Preparation of the Compound

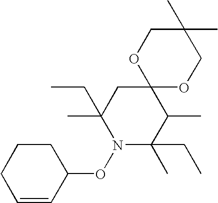

To a stirred mixture of 8.95 g (30 mmol) 8,10-diethyl-3,3,7,8,10-pentamethyl-1,5-dioxa-9-aza-spiro[5.5]undecane-N-oxide, 24.6 g (300 mmol) cyclohexene and 0.11 g (0.3 mmol) tetrabutylammoniumiodide are added at 65° C. within 20 minutes 5.8 g (45 mmol) t-butylhydroperoxid (70% aqueous solution). The temperature is maintained at 65° C. for 15 minutes until all of the N-oxide has reacted. The reaction mixture is cooled down to 25° C. and stirred with 57 g of an aqueous 10% Na₂SO₃ solution until the disappearance of excess t-butylhydroperoxide. The aqueous phase is then separated and washed with cyclohexane. The combined organic phases are washed with brine, dried over MgSO₄, filtered and the solvent distilled off on a rotary-evaporator, yielding 10.5 g (92% of theory) of a slightly orange oil. Purification by Flash-Chromatography (silica gel, Hexane/Ethylacetate 8/2) affords 9.7 g (85% of theory) the title compound as a viscous, colourless oil. Analysis required for C₂₃H₄₁NO₃ (379.58): C, 72.78%, H, 10.89%, N, 3.69%; found: C, 72.61%, H, 10.65%, N, 3.66%.

### EXAMPLE 13

### Preparation of the Compound

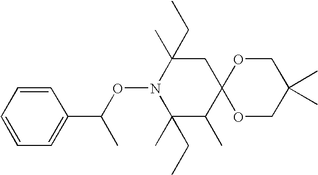

To a stirred mixture of 9.1 g (30 mmol) 8,10-Diethyl-3,3,7,8,10-pentamethyl-1,5-dioxa-9-aza-spiro[5.5]undecane-N-oxide, 31.9 g (300 mmol) Ethylbenzene and 0.11 g (0.3 mmol) Tetrabutylammoniumiodide are added at 60° C. within 25 minutes 5.8 g (45 mmol) t-Butylhydroperoxid (70% aqueous solution). The temperature is maintained at 65° C. for 15 minutes until all of the N-oxide has reacted. The reaction mixture is cooled down to 25° C. and stirred with 57 g of an aqueous 10% Na₂SO₃ solution until the disappearance of excess t-Butylhydroperoxide. The aqueous phase is then separated and washed with Ethylbenzene. The combined organic phases are washed with Brine, dried over MgSO₄, filtered and the solvent distilled off on a rotary-evaporator, yielding 12.4 g (102% of theory) of a slightly yellow oil. Purification by Flash-Chromatography (silica gel, Hexane/Ethylacetate 9.5/0.5) affords 10 g (82.6% of theory) of the title compound as a viscous, colourless oil. Analysis required for C₂₅H₄₁NO₃ (403.61): C, 74.40%, H, 10.24%, N, 3.47%; found: C, 74.29%, H, 10.47%, N, 3.36%.

### EXAMPLE 14

Preparation of the compound of Example 1 with the catalyst Bu₄Nl generated in situ from Bu₄NCl/Nal; yield determination by HPLC.

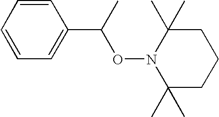

To a stirred mixture of 0.5 g (3.2 mmol) 2,2,6,6-tetramethylpiperidine-N-oxide (TEMPO), 3.8 g (35.6 mmol) ethylbenzene, 0.0092 g (0.032 mmol) tetrabutylammoniumchloride and 0.0048 g (0.032 mmol) sodium iodide dissolved in 1 ml water are added at 50° C. 0.62 g (4.8 mmol) t-butylhydroperoxid (70% aqueous solution). The temperature is maintained at 50° C. for 80 minutes, after which a sample is withdrawn and analyzed by quantitative HPLC. The yield is 78%.

### EXAMPLE 15

Preparation of the compound of Example 12 using immobilized onium iodide. This allows the catalyst be filtered off after the reaction.

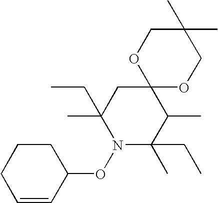

To a stirred mixture of 8.95 g (30 mmol) 8,10-diethyl-3,3,7,8,10-pentamethyl-1,5-dioxa-9-aza-spiro[5.5]undecane-N-oxide, 24.6 g (300 mmol) cyclohexene and 0.3 g (0.3 mmol) tributylmethylammonium iodide bound to polystyrene (1 meq iodide/g) are added at 70° C. within 35 minutes 5.8 g (45 mmol) t-butylhydroperoxid (70% aqueous solution). The temperature is maintained at 70° C. for 18.5 hours until all of the nitroxide has reacted. The reaction mixture is cooled down to 25° C. and the catalyst filtered off. The filtrate is stirred with 57 g of an aqueous 10% Na₂SO₃ solution until the disappearance of excess t-butylhydroperoxide. The aqueous phase is then separated and washed with cyclohexane. The combined organic phases are washed with brine, dried over MgSO₄, filtered and the solvent distilled off on a rotary-evaporator, yielding 10.7 g (94% of theory) of the title product as a slightly orange oil.

### EXAMPLE 16

### Preparation of the Compound of Example 9

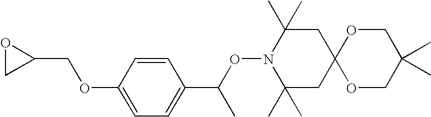

To a stirred mixture of 0.769 g (3 mmol) 3,3,8,8,10,10-hexamethyl-1,5-dioxa-9-aza-spiro[5.5]undecane-N-oxide, 1.6 g (9 mmol, 3 eq) 2-(4-ethyl-phenoxymethyl)-oxirane, 0.046 g (0.3 mmol, 0.1 eq) biphenyl (internal standard) and 0.03 mmol (0.01 eq) onium iodide are added at 60° C. 0.579 g (4.5 mmol, 1.5 eq) t-butylhydroperoxid (70% aqueous solution). The temperature is maintained at 60° C. Samples are withdrawn and analyzed by quantitative HPLC.

Using Bu₄Nl as onium iodide yields 82% of theory after 22 h (nitroxide conversion: 97%). Good results are also achieved when the amount of 2-(4-ethyl-phenoxymethyl)-oxirane is reduced to 2, 1.5 or 1 eq.; or when using 1 eq. of 2-(4-ethyl-phenoxymethyl)-oxirane, the catalyst is replaced by the equivalent amount of Ph₄Pl or Oct₃MeNl, or the amount of Bu₄Nl is increased to 0.15 mmol (0.05 eq.).

### EXAMPLE 17

### Preparation of the Compound

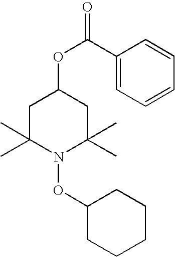

To a stirred mixture of 0.829 g (3 mmol) benzoic acid-2,2,6,6-tetramethyl-piperidin-4-yl-N-oxid ester, 2.53 g (30 mmol, 10 eq) cyclohexane, 0.046 g (0.3 mmol, 0.1 eq) biphenyl (internal standard) and 0.03 mmol (0.01 eq) onium iodide are added at 60° C. 0.579 g (4.5 mmol, 1.5 eq) t-butylhydroperoxid (70% aqueous solution). The temperature is maintained constant. Samples are withdrawn after 22 h and analyzed by quantitative HPLC. Results are given in the tables below:

TABLE
Yield and nitroxide conversion after 22 h reaction at various
temperatures

| | Reaction | Product yield | Nitroxide |
|---|---|---|---|
| Catalyst | Temperature | [%] | conversion [%] |
| Bu₄NI | 60° C. | 33 | 38 |
| Oct₃MeNI | 60° C. | 31 | 35 |
| Bu₄NI | 70° C. | 43 | 48 |
| Bu₄NI | 80° C. | 46 | 52 |

Good results are also achieved when the amount onium iodide catalyst or the amount of tert.butyl hydroperoxide is doubled.

TABLE
Product yield and nitroxide conversion after 22 h reaction at 80° C.
and using 9 mmol (3 eq.) of tert.butyl hydroperoxide

| | Product yield | Nitroxide conversion |
|---|---|---|
| Catalyst | [%] | [%] |
| Bu₄NI | 63 | 69 |
| Oct₄NI | 59 | 67 |
| Hexadecyl₄NI | 59 | 68 |
| Dodecyl₄NI | 58 | 67 |
| Hex₄NI | 58 | 68 |
| Octadecyl₂Me₂NI | 57 | 64 |
| HexadecylBzMe₂NI | 57 | 63 |
| Tetradecyl₂Me₂NI | 56 | 63 |
| Oct₃PrNI | 56 | 65 |
| OctBzMe₂NI | 56 | 63 |
| Oct₃MeNI | 54 | 63 |
| HexadecylPyI | 54 | 59 |
| Oct₂Me₂NI | 53 | 62 |
| OctMe₃NI | 52 | 57 |
| Et₄N | 38 | 42 |
| Oct₂MeSI | 12 | 17 |
| Ph₄PI | 74 | 88 |
| Ph₃iPrPI | 71 | 87 |
| Ph₃EtPI | 63 | 74 |
| Ph₃HexPI | 61 | 71 |
| Bu₄PI | 61 | 68 |
| Bu₃HexadecylPI | 61 | 68 |
| Oct₄PI | 58 | 66 |
| Ph₃MePI | 57 | 65 |
| Ph₂Me₂PI | 51 | 56 |
| Et₄PI | 46 | 50 |
| PhMe₃PI | 39 | 44 |
| Ph₃(CH₂CO₂Me)PI | 36 | 35 |
| Ph₃BzPI | 34 | 40 |

Abbreviations:
Me methyl, Et ethyl, Pr n-propyl, iPr iso-propyl, Bu n-butyl, Hex n-hexyl, Oct n-octyl, Ph phenyl, Bz benzyl, Py 1-pyridinium

Using a wide variety of catalysts, the present process effectively converts the N-oxide into the desired product, yielding only low levels of by-products.

### EXAMPLE 18

### Preparation of the Compound of Example 17 Using Ph₄Pl as Catalyst

To a stirred mixture of 8.3 g (30 mmol) benzoic acid-2,2,6,6-tetramethyl-piperidin-4-yl-N-oxid ester, 25.4 g (300 mmol) cyclohexane and 0.14 g (0.3 mmol) tetraphenylphosphonium iodide are added at 80° C. within 30 minutes 11.6 g (90 mmol) t-butylhydroperoxid (70% aqueous solution). The temperature is maintained at 80° C. for 19.3 hours. The reaction mixture is cooled down to 25° C. and stirred with aqueous 10% Na₂SO₃ solution until the disappearance of excess t-butylhydroperoxide. The aqueous phase is then separated and washed with cyclohexane. The combined organic phases are washed with brine, dried over MgSO₄, filtered and the solvent distilled off on a rotary-evaporator, yielding 9 g of a red oil. Purification by flash-chromatography (silica gel, hexane/ethylacetate 9/1) affords 6.8 g (63% of theory) of the product as a viscous, colorless oil. Analysis required for C₂₂H₃₃NO₃ (359.51): C, 73.50%, H, 9.25%, N, 3.90%; found: C, 72.68%, H, 9.39%, N, 3.85%.

### EXAMPLE 19

### Preparation of the Compound

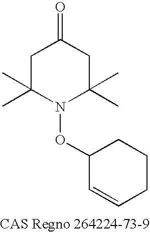

To a stirred mixture of 7.7 g (45 mmol) triacetoneamine-N-oxide, 37.3 g (450 mmol) cyclohexene and 0.17 g (0.45 mmol) tetrabutylammonium iodide are added at 60° C. within 1 hour 17.4 g (135 mmol) t-butylhydroperoxid (70% aqueous solution). The temperature is maintained at 60° C. for 21.7 hours. After further addition of catalyst (0.24 g, 0.45 mmol trioctylmethylammonium iodide) and t-butylhydroperoxide (17.4 g, 135 mmol) the temperature is maintained another 24 hours. The reaction mixture is then cooled down to 25° C. and stirred with aqueous 10% Na₂SO₃ solution until the disappearance of excess t-butylhydroperoxide. The aqueous phase is separated and washed with cyclohexane. The combined organic phases are washed with brine, dried over MgSO₄, filtered and the solvent distilled off on a rotary-evaporator, yielding 11.7 g of an orange oil. Purification by flash-chromatography (silica gel, hexane/ethylacetate 9/1) affords the title product as a colorless oil. Analysis required for C₁₅H₂₅NO₂ (251.37): C, 71.67%, H, 10.02%, N, 5.57%; found: C, 71.33%, H, 10.03%, N, 5.78%.

### EXAMPLE 20

### Preparation of the Compound

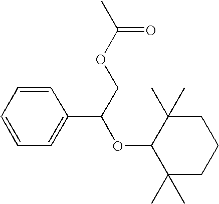

To a stirred mixture of 5 g (32 mmol) TEMPO, 52.5 g (320 mmol) 2-Phenylethylacetate and 0.12 g (0.32 mmol) Tetrabutylammoniumiodide are added at 60° C. within 25 minutes 12.37 g (96 mmol) t-Butylhydroperoxid (70% aqueous solution). The temperature is maintained at 60° C. for 18.67 hours until all of the TEMPO has reacted. The reaction mixture is cooled down to 25° C. and stirred with 121 g of an aqueous 10% Na₂SO₃ solution until the disappearance of excess t-Butylhydroperoxide. The aqueous phase is then separated and washed with Ethylbenzene. The combined organic phases are washed with Brine, dried over MgSO₄, filtered and the solvent distilled off on a rotary-evaporator. The crude product is purified by flash-chromatography (silica gel, Hexane/Ethylacetate 9/1), yielding the title product as a colorless oil. Analysis for C₁₉H₂₉NO₃ (319.45): C, 71.44%, H, 9.15%, N, 4.38%; found: C, 71.36%, H, 9.20%, N, 4.21%. ¹H-NMR (CDCl₁₃), δ (ppm): 0.66 (broad s, 3H), 1.08-1.60 (m, 15H), 1.95 (s, 3H), 4.23-4.30 (m, 1H), 4.57-4.61 (m, 1H), 4.91 (t, J=8 Hz, 1H), 7.28-7.37 (m, 5H).

### EXAMPLE 21

### Preparation of the Compound

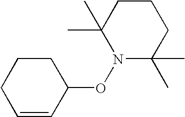

To a stirred mixture of 7.8 g (50 mmol) TEMPO, 41.1 g (500 mmol) Cyclohexene and 0.18 g (0.5 mmol) Tetrabutylammoniumiodide are added at 55° C. within 30 minutes 7.4 g (58 mmol) t-Butylhydroperoxid (70% aqueous solution). The reaction mixture is cooled down to 25° C. and stirred with 63 g of an aqueous 20% Na₂SO₃ solution until the disappearance of excess t-Butylhydroperoxide. The aqueous phase is then separated and washed with Cyclohexane. The combined organic phases are passed through a plug of silica gel and washed with Brine, dried over MgSO₄, filtered and the solvent distilled off on a rotary-evaporator. The crude product is purified by distillation, yielding 8 g (67.4% of theory) of an orange oil (bp 62° C.-65° C./0.04 mbar). Analysis required for C₁₅H₂₇NO (237.39): C, 75.90%, H, 11.46%, N, 5.90%; found: C, 75.69%, H, 11.99%, N 5.75%. ¹H-NMR (CDCl₃), δ (ppm): 1.13-2.07 (m, 24H), 4.24 (br s, 1H), 5.77-5.81 (m, 1H), 5.91-5.95 (m, 1H).

### EXAMPLE 22

### Hydrogenation of the Product of Example 21

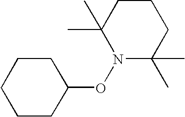

A mixture of 0.95 g (4 mmol) 1-(Cyclohex-2-enyloxy)-2,2,6,6-tetramethyl-piperidine and 0.2 g Pd on charcoal (10%) in 10 ml Methanol is hydrogenated at 25° C. and 4 bar Hydrogen. Filtration and evaporation of the solvent yields the title product as a slightly orange oil. Analysis for C₁₅H₂₉NO (239.40): C, 75.26%, H, 12.21%, N, 5.85%; found: C, 74.53%, H, 12.07%, N, 5.90%. ¹H-NMR (CDCl₃), δ (ppm): 1.12-1.39 (m, 19H), 1.40-1.65 (m, 7H), 1.74 (br s, 1H), 2.04 (br s, 1H), 3.58 (m, 1H).

### EXAMPLE 23

### Hydrogenation of the Crude Product of Example 21

A mixture of the crude product from example 21 (10.87 g, 91.6% of theory) and 2.4 g Pd on charcoal (10%) in 120 ml Methanol is hydrogenated as described in example 22. Filtration and evaporation of the solvent yields 6.8 g of a slightly yellow oil. Analysis required for C₁₅H₂₉NO (239.40): C, 75.26%, H, 12.21%, N, 5.85%; found: C, 74.53%, H, 12.07%, N 5.90%. ¹H-NMR (CDCl₃), δ (ppm): 1.12-1.39 (m, 19H), 1.40-1.65 (m, 7H), 1.74 (br s, 1H), 2.04 (br s, 1H), 3.58 (m, 1H).

### EXAMPLE 24

### Preparation of the Compound

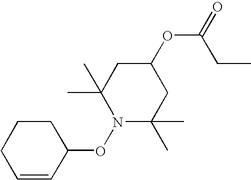

To a stirred mixture of 7.3 g (32 mmol) Propionic acid-2,2,6,6-tetramethylpiperidin-4-yl-N-oxide ester, 26.3 g (320 mmol) Cyclohexene and 0.12 g (0.32 mmol) Tetrabutylammoniumiodide are added at 55° C. within 25 minutes 6.2 g (48 mmol) t-Butylhydroperoxid (70% aqueous solution). The temperature is maintained at 55° C. for 5 minutes until all of the TEMPO has reacted. The reaction mixture is cooled down to 25° C. and stirred with 61 g of an aqueous 10% Na₂SO₃ solution until the disappearance of excess t-Butylhydroperoxide. The aqueous phase is then separated and washed with Cyclohexane. The combined organic phases are passed through a plug of silica gel and washed with Brine, dried over MgSO₄, filtered and the solvent distilled off on a rotary-evaporator, yielding 8.7 g (87.9% of theory) of the above product as a slightly orange oil. Analysis required for C₁₈H₃₁NO₃ (309.45): C, 69.87%, H, 10.10%, N, 4.53%; found: C, 69.36%, H, 10.03%, N, 4.45%. ¹H-NMR (CDCl₃), δ (ppm): 1.12 (t, J=8 Hz, 3H), 1.20-1.26 (m, 12H), 1.52-1.58 (m, 4H), 1.73-2.1 (m, 6H), 2.29 (q, J=8 Hz, 2H), 4.23 (m, 1H), 5.05 (m, 1H), 5.79-5.82 (m, 1H), 5.90-5.94 (m, 1H).

### EXAMPLE 25

### Hydrogenation of the Product of Example 24

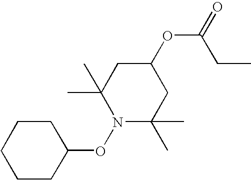

A mixture of CG40-1201 (1 g, 3.19 mmol) and 0.17 g Pd on charcoal (10%) in 30 ml Hexane is hydrogenated as described in example 6. Filtration and evaporation of the solvent yields 0.9 g (90.6% of theory) of a slightly yellow oil. Analysis required for C₁₈H33NO₃ (311.47): C, 69.41%, H, 10.68%, N, 4.50%; found: C, 69.20%, H, 10.76%, N, 4.42%. ¹H-NMR (CDCl₃), δ (ppm): 1.09 (t, J=8 Hz, 3H), 1.10-1.26 (m, 17H), 1.52-1.57 (m, 3H), 1.74-1.84 (m, 4H), 2.03-2.05 (m, 2H), 2.28 (q, J=8 Hz, 2H), 3.56-3.62 (m, 1H), 4.98-5.06 (m, 1H).

### EXAMPLE 26

### Preparation of the Compound

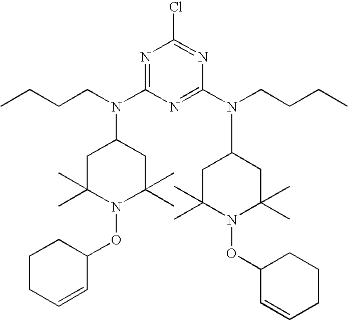

To a stirred mixture of 14.2 g (25 mmol) of N,N′-Dibutyl-6-chloro-N,N′-bis-(2,2,6,6-tetramethyl-piperidin-4-yl-N-oxide)-[1,3,5]-triazine-2,4-diamine, 41 g (500 mmol) Cyclohexene and 0.18 g (0.5 mmol) Tetrabutylammoniumiodide are added at 57° C. within 30 minutes 9.7 g (75 mmol) t-Butylhydroperoxid (70% aqueous solution). The temperature is maintained at 57° C. for 5 minutes until all of the TEMPO has reacted. The reaction mixture is cooled down to 25° C. and stirred with 63 g of an aqueous 10% Na₂SO₃ solution until the disappearance of excess t-Butylhydroperoxide. The aqueous phase is then separated and washed with Cyclohexane. The combined organic phases are washed with Brine, dried over MgSO₄, filtered and the solvent distilled off on a rotary-evaporator, yielding 14.5 g (79.6% of theory) of a slightly yellow solid. Crystallization from Acetone/Hexane yields 12.2 g (67%) of a white solid, mp 83° C.-87° C. Analysis required for C₄₁H₇₀ClN₇O₂ (728.51): C, 67.60%, H, 9.69%, Cl, 4.87%, N, 13.46%; found: C, 67.27%, H, 9.63%, Cl, 4.97%, N, 13.34%. ¹H-NMR (CDCl₃), δ (ppm): 0.89-0.96 (m, 6H), 1.22-1.32 (m, 26H), 1.49-1.56 (m, 12H), 1.73-1.78 (m, 8H), 1.89-2.04 (m, 6H), 3.31-3.32 (m, 4H), 4.24-4.26 (m, 2H), 4.99-5.06 (m, 2H), 5.80-5.83 (m, 2H), 5.92-6.02 (m, 2H).

1. Process for the preparation of an amine ether of a sterically hindered amine by reacting a corresponding sterically hindered aminoxide with an aliphatic hydrocarbon compound, wherein the reaction is carried out in the presence of an organic hydroperoxide and an iodide:

2. Process of claim 1 for the preparation of an amine ether of a sterically hindered amine by reacting a corresponding sterically hindered aminoxide with a hydrocarbon compound, wherein the reaction is carried out in the presence of an organic hydroperoxide and a catalytic amount of an iodide.

3. Process of claim 1, wherein the amine ether is of the formula A 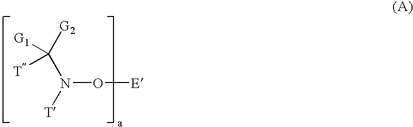
wherein
a is 1 or 2;
when a is 1, E′ is E
when a is 2, E′ is L;
E is C₁-C₃₆ alkyl; C₃-C₁₈ alkenyl; C₂-C₁₈ alkinyl; C₅-C₁₈ cycloalkyl; C₅-C₁₈ cycloalkenyl; a radical of a saturated or unsaturated aliphatic bicyclic or tricyclic hydrocarbon of 7 to 12 carbon atoms; C₂-C₇alkyl or C₃-C₇alkenyl substituted by halogen, C₁-C₈alkoxy or phenoxy; C₄-C₁₂heterocycloalkyl; C₄-C₁₂heterocycloalkenyl; C₇-C₁₅ aralkyl or C₄-C₁₂heteroaralkyl, each of which is unsubstituted or substituted by C₁-C₄ alkyl or phenyl; or E is a radical of formula (VII) or (VIII) 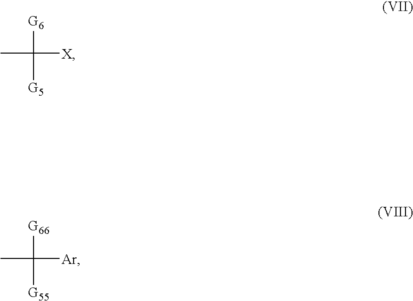
wherein
Ar is C₆-C₁₀aryl or C₅-C₉heteroaryl;
X is phenyl, naphthyl or biphenyl, which is substituted by 1, 2, 3 or 4 D and optionally further substituted by NO₂, halogen, amino, hydroxy, cyano, carboxy, C₁-C₄alkoxy, C₁-C₄alkylthio, C₁-C₄alkylamino or di(C₁-C₄alkyl)amino;
D is a group 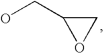
a group C(O)-G₁₃ or a group C(O)-G₉-C(O)-G₁₃;
G₁ and G₂, independently of each other, are hydrogen, halogen, NO₂, cyano, —CONR₅R₆, —(R₉)COOR₄, 13 C(O)—R₇, —OR₈, —SR₈, —NHR₈, —N(R₁₈)₂, carbamoyl, di(C₁-C₁₈alkyl)carbamoyl, —C(═NR₅)(NHR₆), C₁-C₁₈alkyl; C₃-C₁₈alkenyl; C₃-C₁₈alkinyl, C₇-C₉phenylalkyl, C₃-C₁₂cycloalkyl or C₂-C₁₂heterocycloalkyl; C₁-C₁₈alkyl or C₃-C₁₈alkenyl or C₃-C₁₈alkinyl or C₇-C₉phenylalkyl, C₃-C₁₂cycloalkyl or C₂-C₁₂heterocycloalkyl substituted by OH, halogen, NO₂, amino, cyano, carboxy, COOR₂₁, C(O)—R₂₂, C₁-C₄alkoxy, C₁-C₄alkylthio, C₁-C₄alkylamino or di(C₁-C₄alkyl)amino or a group —O—C(O)—R₇; C₂-C₁₈alkyl which is interrupted by at least one O atom and/or NR₅ group; or are C₆-C₁₀aryl; or phenyl or naphthyl which are substituted by C₁-C₄alkyl, C₁-C₄alkoxy, C₁-C₄alkylthio, halogen, cyano, hydroxy, carboxy, COOR₂₁, C(O)—R₂₂, C₁-C₄alkylamino or di(C₁-C₄alkyl)amino; or G₁ and G₂ together with the linking carbon atom form a C₃-C₁₂cycloalkyl radical;
G₅ and G₆ are independently of each other H or CH₃;
G₉ is C₁-C₁₂alkylene or a direct bond;
G₁₃ is C₁-C₁₈alkyl;
G₁₄ is C₁-C₁₈alkyl, C₅-C₁₂cycloalkyl, an acyl radical of an aliphatic or unsaturated aliphatic carboxylic or carbamic acid containing 2 to 18 carbon atoms, an acyl radical of a cycloaliphatic carboxylic or carbamic acid containing 7 to 12 carbon atoms, or acyl radical of an aromatic acid containing 7 to 15 carbon atoms;
G₅₅ is H, CH₃ or phenyl;
G₆₆ is —CN or a group of the formula —COOR₄ or —CONR₅R₆ or —CH₂—O-G₁₄;
L is alkylene of 1 to 18 carbon atoms, cycloalkylene of 5 to 8 carbon atoms, cycloalkenylene of 5 to 8 carbon atoms, alkenylene of 3 to 18 carbon atoms, alkylene of 1 to 12 carbon atoms substituted by phenyl or by phenyl substituted by alkyl of 1 to 4 carbon atoms; or is alkylene of 4 to 18 carbon atoms interrupted by COO and/or phenylene;
T′ is tertiary C₄-C₁₈alkyl or phenyl, each of which are unsubstituted or substituted by halogen, OH, COOR₂₁ or C(O)—R₂₂; or T′ is C₅-C₁₂cycloalkyl; C₅-C₁₂cycloalkyl which is interrupted by at least one O or —NR₁₈—; a polycyclic alkyl radical having 7-18 carbon atoms, or the same radical which is interrupted by at least one O or —NR₁₈—; or T′ is —C(G₁)(G₂)-T″; or C₁-C₁₈alkyl or C₅-C₁₂cycloalkyl substituted by 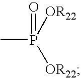
T″ is hydrogen, halogen, NO₂, cyano, or is a monovalent organic radical comprising 1-50 carbon atoms;
or T″ and T′ together form a divalent organic linking group completing, together with the hindered amine nitrogen atom and the quaternary carbon atom substituted by G₁ and G₂, an optionally substituted five- or six-membered ring structure;
and
R₄ is hydrogen, C₁-C₁₈alkyl, phenyl, an alkali metal cation or a tetraalkylammonium cation;
R₅ and R₆ are hydrogen, C₁-C₁₈alkyl, C₂-C₁₈alkyl which is substituted by hydroxy or, taken together, form a C₂-C₁₂alkylene bridge or a C₂-C₁₂-alkylene bridge interrupted by O or/and NR₁₈;
R₇ is hydrogen, C₁-C₁₈alkyl or C₆-C₁₀aryl;
R₈ is hydrogen, C₁-C₁₈alkyl or C₂-C₁₈hydroxyalkyl;
R₉ is C₁-C₁₂alkylene or a direct bond;
R₁₈ is C₁-C₁₈alkyl or phenyl, which are unsubstituted or substituted by halogen, OH, COOR₂₁ or C(O)—R₂₂;
R₂₁ is hydrogen, a alkali metal atom or C₁-C₁₈alkyl; and
R₂₂ is C₁-C₁₈alkyl;
the aminoxide is of formula B 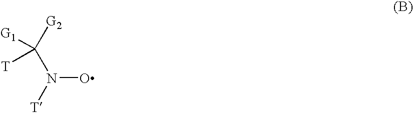
and the hydrocarbon is of formula IV or V
E-H (IV)
H-L-H (V)
wherein E, G₁, G₂, L, T and T′ are as defined for formula A.

4. Process according to claim 1, wherein the organic hydroperoxide is a peroxoalcohol containing 3-18 carbon atoms.

5. Process according to claim 1, wherein 1 to 100 moles of the hydrocarbon, 1 to 20 moles of organic hydroperoxide, and 0.001 mmoles to 0.5 moles of iodide catalyst are used per mole of aminoxide.

6. Process according to claim 1, which is carried out in the absence of copper or a copper compound.

7. Process according to claim 1, wherein the hydrocarbon is used in excess and serves both as reactant and as solvent for the reaction and/or wherein a further inert organic or inorganic solvent is used.

8. Process according to claim 1, wherein the reaction is carried out in the presence of a phase transfer catalyst.

9. Process according to claim 8, wherein the catalyst is selected from alkaline or alkaline earth metal iodides, ammonium iodides and phosphonium iodides.

10. Process according to claim 3, wherein in the formulae A and B T and T′ together are an organic linking group containing 2-500 carbon atoms and 0-200 hetero atoms selected from oxygen, phosphorus, sulfur, silicon, halogen and nitrogen as tertiary nitrogen, and forming, together with the carbon atoms it is directly connected to and the nitrogen atom, an optionally substituted, 5-, 6 or 7-membered cyclic ring structure.

11. Process according to claim 1, wherein the aliphatic hydrocarbon compound contains an ethylenic double bond, and the product is subsequently hydrogenated.

12. (canceled)

13. A compound of the formula a, b c or d

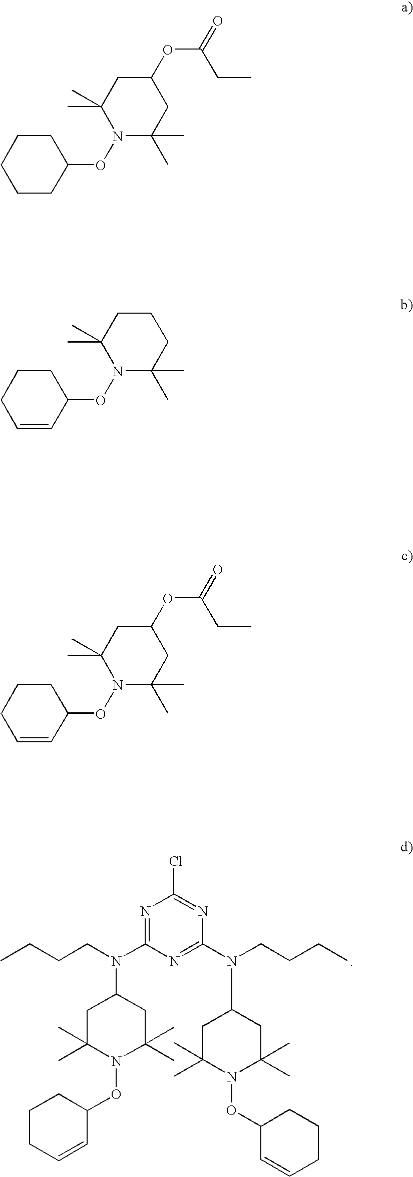

14. (canceled)
